(19) United States
(12) Patent Application Publication
(10) Pub. No.: US 2014/0349938 A1
(43) Pub. Date: Nov. 27, 2014

(54) METHODS OF DIAGNOSING AND TREATING AMYOTROPHIC LATERAL SCLEROSIS

(71) Applicants: Robin Reed, Boston, MA (US); Shi Chen, Malden, MA (US); Yong Yu, Jamaica Plain, MA (US); Tomohiro Yamazaki, Brookline, MA (US)

(72) Inventors: Robin Reed, Boston, MA (US); Shi Chen, Malden, MA (US); Yong Yu, Jamaica Plain, MA (US); Tomohiro Yamazaki, Brookline, MA (US)

(73) Assignee: President and Fellows of Harvard College, Cambridge, MA (US)

(21) Appl. No.: 14/123,392

(22) PCT Filed: Jun. 1, 2012
(86) PCT No.: PCT/US2012/040469

§ 371 Date: Jun. 24, 2014

Related U.S. Application Data
(60) Provisional application No. 61/591,135, filed on Jan. 26, 2012.
(60) Provisional application No. 61/493,233, filed on Jun. 3, 2011.

(51) Int. Cl. G01N 33/68; G01N 33/50

(52) U.S. Cl. CPC G01N 33/6896; G01N 33/6875; G01N 33/5023; G01N 2800/2835; G01N 2800/52; G01N 2800/50
USPC 514/11.5; 435/7.1; 435/6.11; 514/575; 514/17.7; 514/44 R; 514/43; 514/152; 514/9.7; 506/9

(57) ABSTRACT

The invention features methods of diagnosing a subject as having, or at risk of developing ALS by determining the frequency of Gems in cells obtained from the subject. These methods include diagnosing the severity or monitoring the progression of ALS by determining the Gem frequency in a subject. Also, the invention features methods of identifying compounds useful for the treatment of ALS as well as methods for the treatment of ALS.

### CROSS-REFERENCE TO RELATED APPLICATIONS

This application claims benefit of U.S. Provisional Application Nos. 61/493,233 and 61/591,135 filed Jun. 3, 2011, and Jan. 26, 2012, respectively. Each of which is incorporated by reference in its entirety.

### STATEMENT AS TO FEDERALLY FUNDED RESEARCH

This invention was made with Government support under National Institutes of Health award GM043375. The Government has certain rights in the invention.

### BACKGROUND OF THE INVENTION

This invention is in the field of diagnosing and treating amyotrophic lateral sclerosis.

Amyotrophic lateral sclerosis (ALS) is a progressive, fatal neurodegenerative disorder. Its incidence has been reported to be 0.6-2.6/100,000, with a slight male predominance. The disease incidence peaks in the sixth decade of life and survival is typically two to five years. ALS inevitably leads to death from respiratory paralysis in the absence of mechanical ventilation.

Familial cases account for about 10% of ALS. Mutations in cytosolic copper-zinc superoxide dismutase 1 (SOD1) have been shown to account for 20-25% of these familial cases. Mutations in vesicle-associated membrane protein-associated protein (VAPB) have been shown to cause either classical ALS or atypical motor neuron disease in a small number of Brazilian families. A handful of other genes have been implicated in atypical motor neuron disease, including upper-motor-neuron-predominant ALS2 (alsin), juvenile ALS (senataxin), and lower motor neuropathy (DCTN1). A second form of juvenile inherited ALS has been linked to chromosome 15q. In the majority of familial classical ALS cases, however, the causative gene is unknown.

Accordingly, there exists a need in the art for methods of diagnosing ALS and identifying compounds useful for treating ALS.

### SUMMARY OF THE INVENTION

We have shown that FUS, which is mutated in ALS, interacts directly with SMN, the protein deficient in SMA. SMN is a component of the SMN complex, which functions in snRNP biogenesis, and we show that the SMN complex and U1 snRNP are the most abundant factors associated with FUS in nuclear extracts. Functionally, we show that FUS, similar to SMN, is required for formation/stability of nuclear Gems in both HeLa cells and mouse motor neurons. Strikingly, as observed in SMA patient fibroblasts, we found that Gems are deficient in ALS patient fibroblasts carrying FUS mutations. This discovery provides methods for diagnosing and monitoring ALS, as well as methods for identifying compounds that may be useful for the treatment of ALS.

Accordingly, in one aspect, the invention features a method of diagnosing a subject as having, or at risk of developing, ALS by obtaining a sample of non-neuronal cells isolated from the subject and determining the frequency of Gems in the isolated cells, or progeny thereof. In this method, a decreased frequency of Gems is indicative of the subject having, or being at risk of developing, ALS. In this method, a frequency of Gems less than 50% (e.g., less than 40%, 30%, 20%, 10%, or 5%) that of cells isolated from a control subject (or progeny thereof) is indicative of the subject having, or being at risk of developing, ALS. Also, a frequency of Gems of less than, e.g., 5, 4, 3, 2, or 1 per cell is indicative of the subject having, or being at risk of developing, ALS.

In a related aspect, the invention features a method of monitoring a disease state in a subject having ALS by obtaining a sample of non-neuronal cells isolated from the subject and determining the frequency of Gems in the isolated cells, or progeny thereof. In this method, a decreased frequency of Gems is indicative of a worsened disease state and an increased frequency of Gems is indicative of an improved disease state.

We have also discovered the identity of proteins that, along with FUS, are part of the SMN complex. Based on this association, these proteins, and the genes which encode them, are implicated as potential diagnostic markers for ALS.

Thus, the invention also features methods of diagnosing a subject as having ALS by obtaining a sample of cells (e.g., non-neuronal cells) isolated from the subject and determining the activity (e.g., the genotype, expression, or binding activity) of one or more components of the SMN complex (e.g., SMN, Gemin2, Gemin3, Gemin4, Gemin5, Gemin6, Gemin7, Gemin8, and UNRIP), U1 snRNP complex, (e.g., U1-70K, U1A, U1C, U1 snRNA), U2 snRNP complex (e.g., U2 snRNA, the SF3 complex, A′, B″), SR proteins (e.g., SFRS3, SFRS9, SFRS7, and related family members), or other components of the spliceosome. In these methods, a decrease in activity of or presence of a mutation in any of the above components is diagnostic of ALS.

In any of the foregoing methods, the sample of cells can include, e.g., non-neuronal cells selected from the group of epithelial cells, fibroblasts, fibrocytes, myocytes, tendon cells, myocardiocytes, adipocytes, interstitial cells, lymphocytes, gastric chief cells, parietal cells, goblet cells, hepatocytes, urothelial cells, osteocytes, iPS cells, and paneth cells. The cells can be present, e.g., in urine, blood, serum, plasma, saliva, amniotic fluid, or cerebrospinal fluid, or can be isolated from the subject by way of a biopsy.

Also, in either of the foregoing methods, the subject can be selected based on having an allele associated with familial ALS, or a risk of having acquired an allele associated with familial ALS. For example, the diagnostic methods can be performed on subjects having, or at risk of having acquired, a mutation in FUS (e.g., a R521C mutation), TDP43 (e.g., a M337V mutation), superoxide dismutase 1 (SOD1), vesicle-associated membrane protein-associated protein (VAPB), alsin, senataxin, or DCTN1. Alternatively, the diagnostic methods can be performed on subjects not known to have an allele associated with ALS. For example, the diagnostic methods can be used to diagnose sporadic ALS.

In another aspect, the invention features a method of identifying a candidate compound that may be useful to treat ALS, by (a) contacting cells with a candidate compound, (b) comparing the frequency of Gems in the contacted cells with the frequency of Gems in cells not contacted with the candidate compound, and (c) identifying a compound which increases the frequency of Gems in the contacted cells compared to the cells not contacted with the candidate compound; where an increase in the frequency of Gems is indicative of a compound that treats ALS. In this method, the cells can have one or more mutations that reduce the activity of FUS, TDP-43, SMN complex, U1 snRNP complex, U2 snRNP complex, or spliceosome (e.g., SMN, U1-70K, U1A, U1C, U1 snRNA, U2 snRNA, Gemin2, Gemin3, Gemin4, Gemin5, Gemin6, Gemin7, Gemin8, and/or UNRIP) prior to step (a) and/or a reduced frequency of Gems prior to step (a).

In another aspect, the invention features a method of treating ALS in a subject, the method comprising administering to the subject a compound of Table 1 (“a Table 1 compound”) or Table 2 (“a Table 2 compound”), wherein, e.g., the Table 1 or Table 2 compound increases the frequency of Gems relative to control in a suitable assay (such as those described herein, e.g., using human non-neuronal cells such as fibroblasts).

The methods of treating ALS can also include administering a Table 1 or Table 2 compound in a subject having a decreased frequency of Gems. These methods can include, e.g., first determining whether the subject has a decreased frequency of Gems followed by administration of a Table 1 or Table 2 compound if Gems frequency is found to be decreased. Furthermore, the dosing of a Table 1 or Table 2 compound can depend on the frequency of Gems in a subject. For example, if a subject has a decreased frequency of Gems, the subject may be more sensitive to Table 1 and Table 2 compounds and, therefore, require a lower dosage. Alternatively, if a subject has a normal frequency of Gems, then a higher dosage of a Table 1 or Table 2 compound can be administered. In these methods, a frequency of Gems less than 50% (e.g., less than 40%, 30%, 20%, 10%, or 5%) that of cells isolated from a control subject (or progeny thereof), is indicative of the subject having, or being at risk of developing, ALS. Also, a frequency of Gems of less than 5, 4, 3, 2, or 1, per cell is indicative of the subjecting having, or being at risk of developing, ALS.

In any of the foregoing methods, Gem frequency can be determined, e.g., using a confocal microscope or fluorescent microscope. Furthermore, the methods can include contacting the cells with an antibody specific for a Gem protein (e.g., anti-SMN1 (anti-gemin 1), anti-gemin 2, anti-gemin 3, anti-gemin 4, anti-gemin 6, anti-gemin 7, anti STRAP, and anti-TDP43 antibodies) or a probe specific for a Gem snRNA.

Also, in any of the foregoing methods, the frequency of Gems can be the average number of Gems per cell or per nucleus.

In any of the foregoing methods, treatment of ALS can result in improved muscle function, decreased rate of muscle function loss, and/or decreases (or decreased worsening) of the following symptoms: difficulty speaking, slurred speech, and/or difficulty swallowing. Muscle function can be determined by, e.g., EMG. Characteristics of muscle function can include duration of the action potential, the area to amplitude ratio of the action potential, or the number of motor units in the muscle as determined using, e.g., the motor unit number estimation technique.

By “sporadic ALS” is meant the occurrence of ALS in an individual with no known family history of ALS. For example, sporadic ALS occurs absent the presence of an inherited mutation that predisposes an individual to ALS. By “non-neuronal cells” is meant cell populations that do not include neurons or cells derived from neurons cultured in vitro.

As used herein, the term “treat,” “treatment,” or “treating” refers to any method used to partially or completely alleviate, ameliorate, relieve, inhibit, delay onset of, reduce severity of, and/or reduce incidence of one or more symptoms or features of a disease, disorder, and/or condition; in some embodiments, treatment prevents one or more symptoms of features of the disease, disorder, or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### BRIEF DESCRIPTION OF THE FIGURES

FIGS. 1A and 1B are images of western blots showing FUS antibody detects one main band by western (FIG. 1A) and immunoprecipitates one main band (FIG. 1B).

FIG. 1C is an image of a gel showing RNA immunoprecipitated by the indicated antibodies.

FIG. 1D is an image of a western blot showing the immunoprecipitation of the indicated proteins with the indicated antibodies.

FIG. 1E is an image of a western blot showing co-immunoprecipitation of the indicated proteins using the indicated antibodies in the presence and absence of FUS shRNA.

FIG. 1F is an image of a gel showing co-immunoprecipitation of the indicated RNAs using the indicated antibodies in the presence and absence of FUS shRNA.

FIG. 2A is an image of a western blot showing immunoprecipitated FUS from HeLa nuclear extracts using an antibody against FUS.

FIG. 2B is an image of a gel showing total RNA that was extracted from IP and GST pulldown samples used in panel A. 30% input (inpt) was loaded. RNAs were detected with ethidium bromide.

FIG. 2C is an image of a western blot showing the presence of the U1 snRNP proteins, U1-70K, and U1A in the FUS pulldown and immunoprecipitation experiments.

FIG. 2D is a chart showing mass spectrometry data for indicated proteins from FUS IP.

FIG. 2E is an image of a western blot showing immunoprecipitates from HeLa nuclear extracts using FUS, SMN, Gemin 3 (G3), and TDP-43 antibodies, or a negative control antibody, followed by western analysis with the indicated antibodies. The negative control for the FUS and TDP-43 antibodies was a rabbit polyclonal antibody (lane 2, SAP130), and the negative control for the monoclonal antibodies (SMN and Gemin3) was a monoclonal against HA (lane 7). Nuclear extract input is shown in lane 1.

FIG. 2F is an image of a western blot showing immunoprecipitates from HeLa nuclear extracts using FUS in samples treated with RNase prior to immunoprecipitation.

FIG. 2G is an image of a western blot showing gel filtration fractions with indicated antibodies. V: void volume, S: Spliceosome, H: H complex, I: Included volume.

FIG. 3A is a schematic showing FUS and truncated proteins. Gray shading indicates the region where FUS in the nuclear extract binds to GST-FUS. Black shading indicates the FUS truncation containing the RRM, required for FUS binding to U1 snRNP and SMN complex.

FIGS. 3B and 3C are images of Coomassie gels showing GST pulldowns from buffer (−) or nuclear extract (NE) (+) using GST alone, full length (FL) GST-FUS, or the indicated GST-FUS truncated proteins. * in FIG. 3C indicates U1-70K. Molecular weight markers in kD are indicated.

FIG. 3D is an image of an ethidium bromide-stained gel showing total RNA isolated after GST pulldowns using the indicated FUS truncations. SnRNAs and tRNA are shown.

FIG. 3E is an image of a western blot using the indicated antibodies using a portion of the samples shown in FIG. 3C.

FIG. 3F is an image of a western blot showing the indicated purified proteins (2 μg) that were mixed in the presence of RNase followed by GST pulldowns. Proteins were separated on a 4% SDS gradient gel and detected with Coomassie. Markers in kD are indicated.

FIG. 4A is a schematic showing FUS domain structure and truncated proteins

FIGS. 4B and 4C are images of Coomassie-stained gels showing GST pulldowns in RNase-treated conditions using the indicated FUS truncated proteins in the presence of purified His-SMN or His-TDP-43. SMN or TDP-43 (+) or buffer alone (−). * indicates SMN or TDP-43. Molecular weight markers in kD are indicated.

FIG. 5A is series of images generated with immunofluorescent microscopy for SMN in HeLa cells infected with shRNA against FUS. Scrambled shRNA was used as negative control. DAPI staining was used to identify the nucleus.

FIG. 5B is a series of images generated with immunofluorescent microscopy for TDP-43 in HeLa cells transfected with siRNA against TDP-43. Scrambled siRNA was used as negative control. DAPI staining was used to identify the nucleus.

FIG. 5C is a series of images generated with immunofluorescent microscopy for SMN in HeLa cells isolated from FUS −/− mice and wild type littermates.

FIG. 5D is a series of images generated with immunofluorescent microscopy for SMN in HeLa cells transfected with siRNA against proteins in the U1 snRNP complex. Scrambled siRNA was used as negative control. DAPI staining was used to identify the nucleus.

FIG. 6 is a series of images generated with immunofluorescent microscopy for the indicated protein in HeLa cells transfected with siRNA against FUS.

FIG. 7A is a series of images generated with immunofluorescent microscopy using the SMN antibody to detect Gems in fibroblasts from an unaffected individual or ALS patient carrying a FUS R521C mutation. DAPI identifies the nucleus. Scale bar, 20 M. The insets show a higher magnification of the nucleus.

FIG. 7B is a series of images generated with immunofluorescent microscopy using the SMN antibody to detect Gems in fibroblasts from an unaffected individual or ALS patient carrying a TDP43 M337V mutation. DAPI identifies the nucleus. Scale bar, 20 M. The insets show a higher magnification of the nucleus.

FIG. 7C is a graph showing Gem levels in ALS patients and unaffected individuals. The means and standard deviations of Gem number/cell were calculated from three independent experiments. At least 150 cells were observed in each experiment. P-values were calculated by comparison with three controls. * indicates p<0.01. The age (years old) and sex (M: male or F: Female) of the individuals are indicated.

### DETAILED DESCRIPTION OF THE INVENTION

Here, we present several independent lines of evidence that adult ALS and childhood SMA are motor neuron diseases caused by defects in a related molecular pathway. We show that FUS, which is mutated in ALS, interacts directly with SMN, the protein deficient in SMA. SMN is a component of the SMN complex, which functions in snRNP biogenesis, and we show that the SMN complex and U1 snRNP are the most abundant factors associated with FUS in nuclear extracts. Functionally, we show that FUS, similar to SMN, is required for formation/stability of nuclear Gems in both HeLa cells and mouse motor neurons. Strikingly, as observed in SMA patient fibroblasts, we found that Gems are deficient in ALS patient fibroblasts carrying FUS mutations. The invention is described in greater detail below.

In one aspect, the invention features methods of diagnosing a subject as having, or at risk of developing ALS by determining the frequency of Gems in cells obtained from the subject (or progeny cells from the cells obtained from the subject) or the activity of one or more genes identified herein. These methods include diagnosing the severity of or monitoring the progression of ALS by determining the Gem frequency in a subject. Also, the invention features methods of identifying compounds useful for the treatment of ALS, as well as method of treating ALS.

### Determination of Gem Frequency

Gems are nuclear structures between about 0.2 μm and about 2.0 μm in diameter that are involved in small nuclear ribonucleoprotein biogenesis. In the cytoplasm, the SMN complex is known to function in snRNP biogenesis. The SMN complex is present in nuclear Gems, but the function of Gems and the SMN complex contained within the nuclear Gems are not known. Gems can be detected using imaging techniques such as confocal microscopy. Furthermore, cells can be treated with fluorescent or luminescent agents (e.g., anti-FUS, anti-SMN1 (anti-gemin 1), anti-gemin 2, anti-gemin 3, anti-gemin 4, anti-gemin 6, anti-gemin 7, anti STRAP, and anti-TDP43 antibodies or oligonucleotide probes to nucleic acid Gem components, including probes against snRNAs). These agents can be visualized with, e.g., fluorescent microscopy. In order to quantify the number of Gems in a given nucleus, it can be necessary to image Gems in multiple focal planes. For example, Gems can be visualized by taking Z stacks at 0.3 micron steps using widefield microscopy. The Z stacks are then collapsed by a computer to form one combined image, and the Gems counted in each collapsed image. The frequency of Gems can be determined, e.g., using an automated process. For example, MetaMorph® or other image processing software can be used to automatically detect Gem frequency in images of cells. In another example, an automated system to capture Gems images can be fine-tuned for automated Gem counting, resulting in a high throughput procedure that can be useful as a diagnostic and drug-screening marker.

Gem frequency can be determined in a representative sample of cells from a subject. For example, Gem frequency can be expressed as an average number of Gems per nuclei or cell. Therefore, in addition to measuring total Gems in a particular sample, the number of cells and/or nuclei also can be quantified. Gem frequency can be determined using samples derived from a variety of different subject tissues. For example, Gem frequency can be detected in cells isolated from a bodily fluid, including, but not limited to, urine, blood, serum, plasma, saliva, amniotic fluid, or cerebrospinal fluid. Gem frequency can also be detected in particular cell types, including epithelial cells, fibroblasts, fibrocytes, myocytes, tendon cells, myocardiocytes, adipocytes, interstitial cells, lymphocytes, myeloid cells, neurons, gastric chief cells, parietal cells, goblet cells, hepatocytes, urothelial cells, osteocytes, and paneth cells. In a preferred embodiment, the cells are non-neuronal cells. Gems can also be detected in stem cells (e.g., iPS cells derived from ALS patient fibroblasts) and cells derived from stem cells (e.g., motor neurons derived from iPS cells).

### Diagnosis and Monitoring Based on Gem Frequency

The present invention features methods and compositions to predict, diagnose, and stratify subjects at risk for developing ALS. The methods and compositions can include the measurement of Gem frequency in a population of cells isolated from subjects (or progeny thereof). The methods can include measurement of absolute Gem frequency as compared to a reference frequency. For example, a Gem frequency that is less than 5, 4, 3, 2, or 1 per cell or nucleus is considered to be diagnostic of ALS or a risk of developing ALS. A frequency of Gems greater than 3, 4, 5, 6, 7, 8, 9, or 10, can be diagnostic of the absence of ALS or risk of developing ALS.

For diagnoses based on relative Gems frequency, a subject having ALS, or a propensity to develop ALS (e.g., an individual having, or at risk of having an allele associated with familial ALS), will show an alteration (e.g., a decrease of 10%, 20%, 30%, 40%, 50%, 60%, 70%, 80%, 90%, or more), in Gem frequency compared to a normal reference sample or level. A normal reference sample can be, for example, a sample taken from the same subject prior to the development of ALS or a sample from a subject not having ALS or an identified risk of developing ALS (or, e.g., a sample from a subject who is subsequently found to exhibit symptoms of ALS). The normal reference sample can also be age and/or sex matched to the subject being tested.

The diagnostic methods of the invention can be performed, e.g., in subjects determined to have an allele associated with familial ALS or a risk of having acquired an allele associated with familial ALS. For example, the diagnostic methods can be performed on subjects having, or at risk of having acquired, a mutation in FUS (e.g., a R521C mutation) (see, e.g., Vance et al. Science, 323:1208-1211 (2009) and Kwiatkowski et al. Science, 323:1205-1208 (2009)), TDP43 (e.g., a M337V mutation), superoxide dismutase 1 (SOD1), vesicle-associated membrane protein-associated protein (VAPB), alsin, senataxin, and DCTN1. Alternatively, the diagnostic methods can be performed on subjects not known to have an allele associated with ALS. For example, the diagnostic methods can be used to diagnose sporadic ALS.

The diagnostic methods of the invention can also be used to monitor the progress of a subject known to have ALS including a subject undergoing treatment for ALS. In such methods, a relative decrease in Gem frequency is indicative of worsening disease state, and an increase in Gem frequency is indicative of an improved disease state.

### Screening

The invention also features methods of screening for compounds useful in treating ALS. Such methods include contacting a cell with a test compound and comparing the Gem frequency in the contacted cells to Gem frequency in untreated cells. Compounds that increase Gem frequency (e.g., by 25%, 50%, 75%, 100%, 200%, 300%, 400%, 500%, or more) are potentially useful in treating ALS. The cells used in the screening methods of the invention can be cells that exhibit a low frequency of Gems (e.g., a frequency of fewer than 5, 4, 3, 2, or 1 Gems per cell or nucleus) prior to contact with a test compound. For example, the cells can have decreased FUS or TDP-43 activity (e.g., as a result of a mutation in FUS or TDP-43 or introduction of an siRNA to FUS or TDP-43), resulting in decreased Gems frequency. The screening methods can be, e.g., high throughput screening methods. In the screening methods of the invention, Gem frequency can be determined as indicated above.

In general, compounds capable of increasing Gem frequency are identified from large libraries of both natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to increase Gems frequency, further fractionation of the positive lead extract is necessary to isolate chemical constituents responsible for the observed effect. Thus, the goal of the extraction, fractionation, and purification process is the careful characterization and identification of a chemical entity within the crude extract that increases Gems frequency. Methods of fractionation and purification of such heterogeneous extracts are known in the art. If desired, compounds shown to be useful as therapeutics for the treatment or prevention of ALS are chemically modified according to methods known in the art.

Diagnosis and Monitoring based on Expression Profiling and Mutational Analysis of Candidate Genes Associated with ALS

The present invention features methods and compositions to predict, diagnose, and stratify subjects at risk for developing ALS. The methods and compositions can include an analysis of aberrant expression and/or mutations of genes associated with ALS, including VCP, OPTN, FIG4, DAO, TDP-43, FUS, C9ORF72, and SOD1 and components of the SMN complex, U1 snRNP complex, and U2 snRNP complex, which have been shown to associate with FUS (e.g., SMN, Gemin2, Gemin3, Gemin4, Gemin5, Gemin6, Gemin7, Gemin8, and UNRIP). The components of the U1 snRNP complex include: U1 snRNA, the proteins U170K, U1A, U1C and associated SR protein, SFRS1. Several others proteins (e.g., SFRS3, SFRS9, SFRS7, and related family members) also associate with FUS and are therefore genes associated with ALS or cause susceptibility to ALS and/or affect disease progression. The methods of the invention can include identification of aberrant expression and/or mutations in above genes. Examples of such methods can include a genetic screen, used to identify mutations in genes resulting in phenotypic changes in Gem frequency, i.e., a decrease in Gem frequency (e.g., mutations in the above genes). Another example is expression profiling, e.g., DNA microarray, and serial analysis of gene expression (SAGE and superSAGE), to measure activity of the above genes in subjects identified as having ALS compared to subjects not having ALS.

For diagnoses based on expression profiles of genes associated with ALS and components of the SMN complex, a subject having ALS, or a propensity to develop ALS (e.g., an individual having, or at risk of having, ALS), will show an alteration (e.g., a decrease of 10%, 20%, 30%, 40%, 50%, 60%, 70%, 80%, 90%, or more), in expression of such genes. A normal reference sample can be, for example, a sample taken from the same subject prior to the development of ALS or a sample from a subject not having ALS or an identified risk of developing ALS (or, e.g., a sample from a subject who is subsequently found to exhibit symptoms of ALS). The normal reference sample can also be age and/or sex matched to the subject being tested. For diagnoses based on mutations of genes associated with ALS and components of the SMN complex, a subject having ALS, or a propensity to develop ALS (e.g., an individual having, or at risk of having, ALS), will show at least one mutation (e.g. 1, 2, 3, 4, 5, or more), in at least one (e.g. 1, 2, 3, 4, 5, or more) of the genes, as determined by sequencing and alignment to a normal reference.

The diagnostic methods of the invention can also be used to monitor the progress of a subject known to have ALS including a subject undergoing treatment for ALS. In such methods, the number of mutations in genes associated with ALS may correlate to a further decrease in Gem frequency, which is an indication of the severity of the disease state. In another aspect, improvements to the disease state may correlate with increased expression in the candidate genes mentioned above.

### Indication and Treatment of ALS Based on Gem Frequency

We have discovered that SMA and ALS share a Gem phenotype, indicating that drug candidates identified for SMA may also be efficacious for ALS. Consequently, the invention also features the treatment of ALS with one or more compounds of identified as being useful for the treatment of SMA, including those compounds listed in Table 1 or Table 2. Desirably, in one of the assays described herein, the compound or compounds increase the frequency of Gems in the contacted cells compared to control cells not contacted with the particular compound of Table 1 or Table 2.

TABLE 1
Vorinostat, suberoylanilide hydroxamic acid, SAHA (N-hydroxy-N′-
phenyl-octanediamide)
Romidepsin ((1S,4S,7Z,10S,16E,21R)-7-ethylidene-4,21-diisopropyl-2-
oxa-12,13-dithia-5,8,20,23-tetrazabicyclo[8.7.6]tricos-16-ene-
3,6,9,19,22-pentone)
Panobinostat, LBH589 ((2E)-N-hydroxy-3-[4-({[2-(2-methyl-1H-indo1-3-
yl)ethyl]amino}methyl)phenyl]acrylamide)
Belinostat, PXD101 ((2E)-N-Hydroxy-3-[3-(phenylsulfamoyl)phenyl]
prop-2-enamide)
Mocetinostat, MGCD0103 (N-(2-Aminophenyl)-4-[[(4-pyridin-3-
ylpyrimidin-2-yl)amino]methyl]benzamide)
PCI-24781 (3-(dimethylaminomethyl)-N-[2-[4-(hydroxycarbamoyl)
phenoxy]ethyl]-1-benzofuran-2-carboxamide)
SB939 ((E)-3-[2-butyl-1-[2-(diethylamino)ethyl]benzimidazol-5-yl]-N-
hydroxyprop-2-enamide)
Givinostat, ITF2357 ({6-[(diethylamino)methyl]naphthalen-2-yl]methyl [4-
(hydroxycarbamoyl)phenyl]carbamate)
Sodium butyrate
Sodium phenylbutyrate
Valproic acid (2-propylpentanoic acid)
CUDC-101 (7-[4-(3-ethynylanilino)-7-methoxyquinazolin-6-yl]oxy-N-
hydroxyheptanamide)
AR-42 (N-hydroxy-4-[[(2S)-3-methyl-2-phenylbutanoyl]amino]benzamide)
Sulforaphane (1-isothiocyanato-4-methylsulfinylbutane)
Kevetrin (4-isothioureidobutyronitrile)
5-azacytidine
Carboxycinnamic acid bis-hydroxamide, CBHA (N-hydroxy-3-[(E)-3-
(hydroxyamino)-3-oxoprop-1-enyl]benzamide)
Suberohydroxamic acid, SBHA (N,N′-dihydroxyoctanediamide)
Entinostat, MS-275 (Pyridin-3-ylmethyl N-[[4-[(2-aminophenyl)
carbamoyl]phenyl]methylicarbamate)
Trichostatin A (7-[4-(dimethylamino)phenyl]-N-hydroxy-4,6-dimethyl-7-
oxohepta-2,4-dienamide)
Hydroxyurea
Resveratrol (5-[(E)-2-(4-hydroxyphenyl)ethenyl]benzene-1,3-diol)
Curcumin ((1E,6E)-1,7-bis(4-hydroxy-3-methoxyphenyl)hepta-1,6-diene-
3,5-dione)
Prolactin
Salbutamol, albuterol (4-[2-(tert-butylamino)-1-hydroxyethyl]-2-
(hydroxymethyl)phenol)
Sodium orthovanadate (trisodium trioxido(oxo)vanadium)
Aclarubicin (methyl (1R,2R,4S)-4-[(2R,4S,5S,6S)-4-(dimethylamino)-5-
[(2S,4S,5S,6S)-4-hydroxy-6-methyl-5-[(2R,6S)-6-methyl-5-oxooxan-2-yl]
oxyoxan-2-yl]oxy-6-methyloxan-2-yl]oxy-2-ethyl-2,5,7-
trihydroxy-6,11-dioxo-3,4-dihydro-1H-tetracene-1-carboxylate)
Ceftriaxone (6R,7R)-7-[[(2Z)-2-(2-amino-1,3-thiazol-4-yl)-2-
methoxyiminoacetyl]amino]-3-[(2-methyl-5,6-dioxo-1H-1,2,4-triazin-3-yl)
sulfanylmethyl]-8-oxo-5-thia-1-azabicyclo[4.2.0]oct-2-ene-2-
carboxylic acid
Indoprofen (2-[4-(3-oxo-1H-isoindo1-2-yl)phenyl]propanoic acid)
Follistatin
Riluzole (6-(trifluoromethoxy)benzothiazol-2-amine)
Rifalazil (3′-Hydroxy-5′-(4-isobutylpiperazinyl)benzoxazinorifamycin)
Thyrotropin-releasing hormone
Levetiracetam ((S)-2-(2-oxopyrrolidin-1-yl)butanamide)
Goserelin (N-(21-((1H-indo1-3-yl)methyl)-1,1-diamino-12-(tert-
butoxymethyl)-6-(2-(2-carbamoylhydrazinecarbonyl)cyclopentane-
carbonyl)-15-(4-hydroxybenzyl)-18-(hydroxymethyl)-25-(1H-
imidazol-5-yl)-9-isobutyl-8,11,14,17,20,23-hexaoxo-
2,7,10,13,16,19,22-heptaazapentacos-1-en-24-yl)-5-oxopyrrolidine-2-
carboxamide)
Dutasteride ((5α,17β-N-{2,5 bis(trifluoromethyl)phenyl}-3-oxo-4-
azaandrost-1-ene-17-carboxamide)
M344 (4-Dimethylamino-N-(6-hydroxycarbamoylhexyl)-benzamide)
CHR-3996 (2-(6-{[(6-fluoroquinolin-2-yl)methyl]amino}bicyclo[3.1.0]
hex-3-yl)-N-hydroxypyrimidine-5-carboxamide)
CG200745 ((E)-N(1)-(3-(dimethylamino)propyl)-N(8)-hydroxy-2-
((naphthalene-1-loxy)methyl)oct-2-enediamide)
Olesoxime (cholest-4-en-3-one oxime)
ISIS-SMNRx (2′-O-2-methyoxyethyl-modified antisense oligonucleotides)

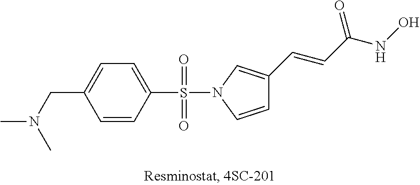 [SMILES: CN(C)Cc1ccc(S(=O)(=O)n2ccc(/C=C/C(=O)NO)c2)cc1]

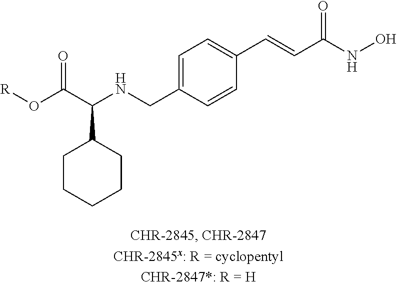

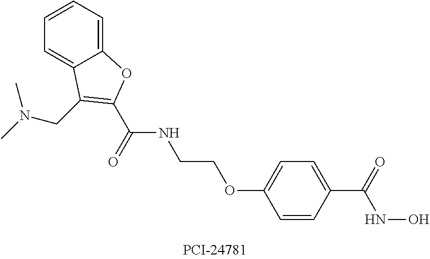 [SMILES: CN(C)Cc1c(C(=O)NCCOc2ccc(C(=O)NO)cc2)oc2ccccc12]

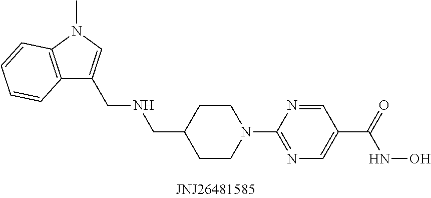 [SMILES: Cn1cc(CNCC2CCN(c3ncc(C(=O)NO)cn3)CC2)c2ccccc21]

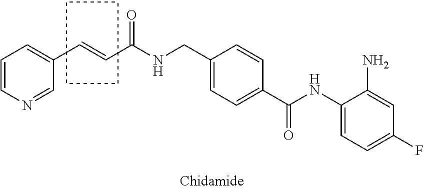 [SMILES: Nc1cc(F)ccc1NC(=O)c1ccc(CNC(=O)/C=C/c2cccnc2)cc1]

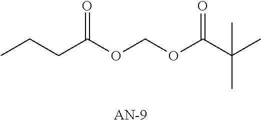

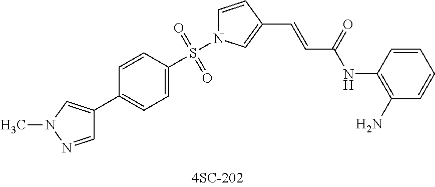 [SMILES: Cn1cc(-c2ccc(S(=O)(=O)n3ccc(/C=C/C(=O)Nc4ccccc4N)c3)cc2)cn1]

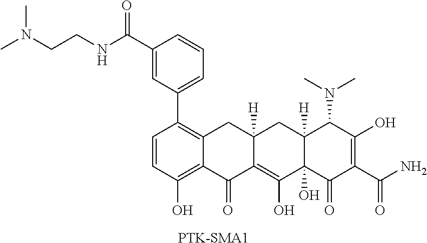

Any of the compounds identified in the following Patents, Applications, or
PCT applications, each of which are incorporated by reference in their
entirety:
PCT/US2010/034838
US20020102614A1
US20030049627A1
US20040023944A1
US20040029903A1
US20040029922A1
US20040044063A1
US20040105849A1
US20050009815A1
US20050026915A1
US20050065173A1
US20050100923A1
US20050171206A1
US20050282890A1
US20050288314A1
US20060088842A1
US20060088873A1
US20060089490A1
US20070031418A1
US20070037869A1
US20070166737A1
US20070207144A1
US20070238747
US20070269834A1
US20070292408A1
US20080096920
US20080187512A1
US20080188433A1
US20080194692
US20090031435A1
US20090042900
US20090076130A1
US20090118269A1
US20090156789A1
US20090182029
US20090312323
US20090312323A1
US20100087511A1
US20100136678A1
US20100143961A1
US20100152204A1
US20100216238A1
US20100233688A1
US20100234402A1
US20100256401A1
US20100267712
US20100267712A1
US20100279893A1
US20100324046
US20110033861A1
US20110086833A1
US20110151445A1
US20110172284
US20110183988
US20110190216A1
US20110207706
US20110218221
US20110223149
US20110224294
US20110269820A1
US20110288168A1
US20110294226A1
US20110319353A1
US20120010178
U.S. Pat. No. 4,983,602
U.S. Pat. No. 5,055,608
U.S. Pat. No. 5,369,108
U.S. Pat. No. 5,608,108
U.S. Pat. No. 5,700,811
U.S. Pat. No. 5,882,868A
U.S. Pat. No. 5,932,616
U.S. Pat. No. 6,020,127A
U.S. Pat. No. 6,040,142A
U.S. Pat. No. 6,080,577A
U.S. Pat. No. 6,087,367
U.S. Pat. No. 6,245,963B1
U.S. Pat. No. 6,376,508B1
U.S. Pat. No. 6,429,011B1
U.S. Pat. No. 6,511,990
U.S. Pat. No. 6,573,300B2
U.S. Pat. No. 6,646,113B1
U.S. Pat. No. 6,924,102B2
U.S. Pat. No. 7,033,752B1
U.S. Pat. No. 7,057,057B2
U.S. Pat. No. 7,084,156B2
U.S. Pat. No. 7,193,105B2
U.S. Pat. No. 7,211,593
U.S. Pat. No. 7,214,831
U.S. Pat. No. 7,446,109
U.S. Pat. No. 7,459,309B2
U.S. Pat. No. 7,579,372B2
U.S. Pat. No. 7,772,238
U.S. Pat. No. 7,795,304
U.S. Pat. No. 7,838,657B2
U.S. Pat. No. 7,842,727B2
U.S. Pat. No. 7,875,432B2
U.S. Pat. No. 7,928,127
U.S. Pat. No. 7,928,136
U.S. Pat. No. 7,935,687
U.S. Pat. No. 7,943,652
U.S. Pat. No. 7,947,830
U.S. Pat. No. 7,960,415
U.S. Pat. No. 7,981,874
U.S. Pat. No. 7,985,755
U.S. Pat. No. 7,994,357B2
U.S. Pat. No. 8,008,344
U.S. Pat. No. 8,013,014
U.S. Pat. No. 8,013,170
U.S. Pat. No. 8,022,041
U.S. Pat. No. 8,022,234
U.S. Pat. No. 8,022,246
U.S. Pat. No. 8,026,260
U.S. Pat. No. 8,044,097
U.S. Pat. No. 8,067,425
U.S. Pat. No. 8,067,568B2
U.S. Pat. No. 8,071,549
U.S. Pat. No. 8,076,086
U.S. Pat. No. 8,076,449
U.S. Pat. No. 8,080,682
U.S. Pat. No. 8,093,392
U.S. Pat. No. 8,097,611
U.S. Pat. No. 8,097,619
U.S. Pat. No. 8,101,616
USRE43,003
W01996012016A1
W02001027085A1
W02001066129A1
W02003045313A2
W02003045313A3
W02003045920A1
W02003099272A1
W02003099760A1
W02003099789A1
W02004047766A2
W02004047766A3
W02004113305A2
W02004113305A3
W02005072720A1
W02005123724A1
W02006116609A2
W02006116609A3
W02006116609A8
W02007002390A2
W02007002390A3
W02007097980A2
W02007097980A3
W02007097980A9
W02007109211A2
W02007109211A3
W02007109211A8
W02008011074A2
W02008011074A3
W02008016973A1
W02008095356A1
W02008095357A1
W02008127722A1
W02008150509A1
W02008157753A1
W02009042907A1
W02009146033A2
W02009146033A3
W02009151546A2
W02009151546A3
W02010014746A1
W02010019236A1
W02010048273A2
W02010048273A3
W02010084136A1
W02010115993A1
W02010120820A1
W02010148249A1
W02010151638A1
W02011032109A1
W02011113889A1
W02011130515A1

TABLE 2
5-(3-Chlorobenzyloxy)quinazoline-2,4-diamine
5-(2-Chlorobenzyloxy)quinazoline-2,4-diamine
5-(2-Methylbenzyloxy)quinazoline-2,4-diamine
5-(2-p-Tolylethoxy)quinazoline-2,4-diamine
5-[2-(4-Chlorophenyl)ethoxy]quinazoline-2,4-diamine
5-(3-Methylbenzyloxy)quinazoline-2,4-diamine
5-(Pyridin-3-ylmethoxy)quinazoline-2,4-diamine
5-(1-Phenylethoxy)quinazoline-2,4-diamine
5-(Cyclohex-3-enylmethoxy)quinazoline-2,4-diamine
5-(Cyclobutylmethoxy)quinazoline-2,4-diamine
5-(2-Methoxyethoxy)quinazoline-2,4-diamine
5-(Cyclopropylmethoxy)quinazoline-2,4-diamine
5-(Cyclohexylmethoxy)quinazoline-2,4-diamine
5-(Cyclopentylmethoxy)quinazoline-2,4-diamine
5-(2-Allyloxyethoxy)quinazoline-2,4-diamine
5-(1-Methylpiperidin-3-ylmethoxy)quinazoline-2,4-diamine
5-(Furan-2-ylmethoxy)quinazoline-2,4-diamine
5-(Thiophen-2-ylmethoxy)quinazoline-2,4-diamine
5-(4-Methylbenzyl)quinazoline-2,4-diamine
5-Benzylquinazoline-2,4-diamine
5-(4-Chlorobenzyl)quinazoline-2,4-diamine
5-(4-Methoxybenzyl)quinazoline-2,4-diamine
5-[3-(4-Chlorophenyl)propoxy]quinazoline-2,4-diamine I
5-[1-(3-Chlorophenyl)ethoxy]quinazoline-2,4-diamine
5-(4-Chlorobenzylsulfanyl)quinazoline-2,4-diamine
5-p-Tolylethynylquinazoline-2,4-diamine
5-(4-Chlorobenzenesulfonyl)quinazoline-2,4-diamine
N-[2-Acetylamino-5-(4-chlorobenzyloxy)quinazolin-4-yl]acetamide
5-(3-Methyl-4,5-dihydroisoxazol-5-ylmethoxy)quinazoline-2,4-diamine
5-(Furan-3-ylmethoxy)quinazoline-2,4-diamine
5-Benzyloxyquinazoline-2,4-diamine
5-(Pyridin-2-ylmethoxy)quinazoline-2,4-diamine
5-Phenethyloxyquinazoline-2,4-diamine
5-0ctyloxyquinazoline-2,4-diamine
N-5-Cyclooctylquinazoline-2,4,5-triamine
5-(Indan-2-yloxy)quinazoline-2,4-diamine
5-((S)-Indan-1-yloxy)quinazoline-2,4-diamine
5-((S)-I-Phenylethoxy)quinazoline-2,4-diamine
5-(4-Chlorophenoxymethyl)quinazoline-2,4-diamine
5-p-Tolyloxymethylquinazoline-2,4-diamine
5-(4-Fluorophenoxymethyl)quinazoline-2,4-diamine
5-Thiophen-3-ylmethylquinazoline-2,4-diamine
5-(Thiophen-3-ylmethoxy)quinazoline-2,4-diamine
5-(1-Pyridin-4-ylethoxy)quinazoline-2,4-diamine
5-[1-(4-Chlorophenyl)ethoxy]quinazoline-2,4-diamine
5-[1-(4-Chlorophenyl)propoxy]quinazoline-2,4-diamine
5-[1-(4-Chlorophenyl)-2,2-dimethylpropoxy]quinazoline-2,4-diamine
5-Benzhydryloxyquinazoline-2,4-diamine
5-(5-Methylisoxazol-3-ylmethoxy)quinazoline-2,4-diamine
5-(Benzo[I,3]dioxol-5-ylmethoxy)quinazoline-2,4-diamine
5-(Tetrahydropyran-2-ylmethoxy)quinazoline-2,4-diamine
5-((R)-I-Phenylethoxy)quinazoline-2,4-diamine
5-(1-Pyridin-2-ylethoxy)quinazoline-2,4-diamine
5-(1-Thiazol-2-ylethoxy)quinazoline-2,4-diamine
5-(Piperidin-1-yl)quinazoline-2,4-diamine
5-(Toluene-3-sulfonyl)-quinazoline-2,4-diamine
5-(6-Chloro-indan-1-yloxy)-quinazoline-2,4-diamine
5-(4-Bromobenzyloxy)-quinazoline-2,4-diamine
5-[1-(3-Iodophenyl)-ethoxy]-quinazoline-2,4-diamine
5-(1-Benzo[1,3]dioxol-5-yl-ethoxy)-quinazoline-2,4-diamine
5-(3,4-Dimethoxybenzyloxy)-quinazoline-2,4-diamine
5-[I-(3-Methoxyphenyl)-ethoxy]-quinazoline-2,4-diamine
5-[1-(3,5-Dimethoxyphenyl)-ethoxy]-quinazoline-2,4-diamine
5-[2-(4-Chlorophenyl)-3-methoxymethoxypropoxy]-quinazoline-2,4diarnine
2-(4-Chlorophenyl)-3-(2,4-diaminoquinazolin-5-yloxy)-propan-1-ol
[4,5-Dichloro-2-(2,4-diaminoquinazolin-5-yloxymethyl)phenyl]methanol
5-(4-Chloro-2-methoxyphenoxy)-quinazoline-2,4-diamine
5-(7-Methoxy-2,3-dihydrobenzofuran-3-yoloxy)-quinazoline-2,4-diarnine
5-(Adamantan-1-ylmethoxy)-quinazoline-2,4-diamine
5-(2-Bromo-benzyloxy)-quinazoline-2,4-diamine
5-(2-Iodo-benzyloxy)-quinazoline-2,4-diamine
5-(3-Bromobenzyloxy)quinazoline-2,4-diamine
5-(3-Iodo-benzyloxy)-quinazoline-2,4-diamine
5-[I-(3,4-Dichlorophenyl)-ethoxy]-quinazoline-2,4-diamine
5-(3,5-Difluorobenzyloxy)quinazoline-2,4-diamine
5-(4-Fluoroindan-1-yloxy)-quinazoline-2,4-diamine
5-(6-Fluoroindan-1-yloxy)-quinazoline-2,4-diamine
5-[1-(2,6-Difluorophenyl)-ethoxy]-quinazoline-2,4-diamine
5-(2,3,5-Trifluorobenzyloxy)-quinazoline-2,4-diamine
5-(2,5-Difluorobenzyloxy)-quinazoline-2,4-diamine
5-(2,4-Difluorobenzyloxy)-quinazoline-2,4-diamine
5-(2,6-Difluorobenzyloxy)-quinazoline-2,4-diamine
5-(3,4-Difluorobenzyloxy)quinazoline-2,4-diamine
5-(5-Chloro-2-methoxybenzyloxy)quinazoline-2,4-diamine
[4-Chloro-2-(2,4-diamino-quinazolin-5-yloxymethyl)-phenyl]-methanol
5-Thiophen-3-yl-quinazoline-2,4-diamine
5-(3-Chlorophenyl)-quinazoline-2,4-diamine
5-[(R)-I-(3-Chlorophenyl)ethoxy]quinazolin-2,4-diamine
5-[1-(3-Fluorophenyl)-ethoxy]-quinazoline-2,4-diamine
5-[1-(2-Trifluoromethylphenyl)-ethoxy]-quinazoline-2,4-diamine
5-[1-(3-Trifluoromethylphenyl)-ethoxy]-quinazoline-2,4-diamine
5-(2-Fluorobenzyloxy)-quinazoline-2,4-diamine
5-(4-Fluorobenzyloxy)-quinazoline-2,4-diamine
5-(3-Trifluoromethylbenzyloxy)-quinazoline-2,4-diamine
5-(2-Trifluoromethylbenzyloxy)-quinazoline-2,4-diamine
5-(4-Trifluoromethylbenzyloxy)-quinazoline-2,4-diamine
5-[1-(4-fluorophenyl)-I-methyl-ethoxy]-quinazoline-2,4-diamine
5-(3-Fluorobenzyloxy)quinazoline-2,4-diamine
5-[1-(2-Fluorophenyl)-ethoxy]-quinazoline-2,4-diamine
5-[1-(2-Chlorophenyl)-ethoxy]-quinazoline-2,4-diamine
5-[1-(4-Trifluoromethylphenyl)ethoxy]quinazoline-2,4-diamine
5-(3,5-Dichlorobenzyloxy)quinazoline-2,4-diamine
5-[1-(3,5-Difluorophenyl)ethoxy]quinazoline-2,4-diamine
5-((S)-I-Naphthalen-1-yl-ethoxy)-quinazoline-2,4-diamine
5-((S)-I-Naphthalen-2-yl-ethoxy)-quinazoline-2,4-diamine
5-((R)-I-Naphthalen-1-yl-ethoxy)-quinazoline-2,4-diamine
5-(1-Naphthalen-1-yl-ethoxy)-quinazoline-2,4-diamine
5-(Quinolin-3-ylmethoxy)-quinazoline-2,4-diamine
5-(Quinolin-8-ylmethoxy)-quinazoline-2,4-diamine
5-[1-(4-Chlorophenyl)-2-methoxyethoxy]-quinazoline-2,4-diamine
(4-Chlorophenyl)-(2,4-diamino-quinazolin-5-yloxy)-acetic acid
5-(Piperidin-4-ylmethoxy)-quinazoline-2,4-diamine
5-(I-Methyl-piperidin-2-ylmethoxy)-quinazoline-2,4-diamine
5-((1R,2R,4S)-Bicyclo [2.2.1]hept-2-yloxy)quinazoline-2,4-diamine
5-(Adamanta-2-yloxy)quiazoline-2,4-diamine
5-(I-Cyclopentyl-ethoxy)-quinazoline-2,4-diamine
4-(2,4-Diamino-quinzaolin-5-yloxymethyl)-piperidin-1-carboxylic acid tert-butyl ester
5-(Bicyclo [2.2.1]hept-5-en-2-ylmethoxy)-quinazoline-2,4-diamine
(4-Chlorophenyl)-[4-(2,4-diamino-quinazolin-5-yloxymethyl)-piperidin-1yl]-methanone
5-(Bicyclo [2.2.1]hept-2-yloxy)-quinazoline-2,4-diamine
5-(I-Cyclohexyl-butoxy)-quinazoline-2,4-diamine
5-(I-Cyclohexyl-ethoxy)-quinazoline-2,4-diamine
5-(3-Methyl-oxetan-3-ylmethoxy)-quinazoline-2,4-diamine
5-(5-Chloro-2,3-dihydro-benzofuran-3-yloxy)-quinazoline-2,4-diamine
5-(I-Cyclohexylpropoxy)-quinazoline-2,4-diamine
5-((1S,2R,5S)-6,6-Dimethylbicyclo [3.1.1]hept-2ylmethoxy)quinazoline-2,4-diamine
5-(2,4-Diamino-quinazolin-5-yloxymethyl)-bicyclo [2.2.1]heptane-2,3-diol
5-[1-(3,4-Dichlorobenzyl)-piperidin-4-ylmethoxy]-quinazoline-2,4 diarnine
(2-Chlorophenyl)-[4-(2,4-diamino-quinazolin-5-yloxymethyl)-piperidin-1-yl]-methanone
(3-Chlorophenyl)-[4-(2,4-diamino-quinazolin-5-yloxymethyl)-piperidin-1-yl]-methanone
[4-(2,4-Diaminoquinzolin-5-yloxymethyl)piperidin-1-yl]-(3iodophenyl)methanone
[4-(2,4-Diaminoquinzolin-5-yloxymethyl)piperidin-1-yl]-(4iodophenyl)methanone
[4-(2,4-Diaminoquinzolin-5-yloxymethyl)piperidin-1-yl]-(2-iodophenyl)methanone
5-(2-Chlorophenoxymethyl)quinazoline-2,4-diamine
5-(4-Chloro-2-methylphenoxymethyl)quinazoline-2,4-diamine
5-[1-(3-Chlorophenyl)-1-methylethoxy]quinazoline-2,4-diamine
5-(4-Chloro-3-methylphenoxymethyl)quinazoline-2,4-diamine
5-(2-Methoxybenzyloxy)quinazoline-2,4-diamine
5-(3-Methoxybenzyloxy)quinazoline-2,4-diamine
5-(4-Methoxybenzyloxy)quinazoline-2,4-diamine
5-[1-(3-Chlorophenyl)cyclohexyloxy]quinazoline-2,4-diamine
5-[1-(3-Chlorophenyl)cyclopropoxy]quinazoline-2,4-diamine
5-(2,4-Difluorophenoxymethyl)quinazoline-2,4-diamine
5-(4-Methoxyphenoxymethyl)quinazoline-2,4-diamine
5-((S)-6-Chloroindan-1-yloxy)quinazoline-2,4-diamine
5-((R)-6-Chloroindan-1-yloxy)quinazoline-2,4-diamine
5-(Bicyclo [2.2.1]hept-2-ylmethoxy)quinazoline-2,4-diamine
5-((1S,2S,5S)-6,6-Dimethylbicyclo [3.1.1]hept-2ylmethoxy)quinazoline-2,4-diamine
5-[1-(4-Fluorophenyl)-2-methoxymethoxyethoxy]quinazoline-2,4diarnine
2-(2,4-Diaminoquinazolin-5-yloxy)-2-(4-fluorophenyl)ethanol
5-(2-Benzo[1,3]dioxol-5-yl-2-methoxymethoxyethoxy)quinazoline-2,4-diarnine
5-(1-Benzo[1,3]dioxol-5-yl-2-methoxymethoxyethoxy)quinazoline-2,4-diarnine
2-(2,4-Diaminoquinazolin-5-yloxy)-1-phenyl-ethanol
5-(3-Chlorophenoxymethyl)quinazoline-2,4-diamine
5-(2-Iodophenoxymethyl)quinazoline-2,4-diamine
1-(4-Chlorophenyl)-2-(2,4-diaminoquinazolin-5-yloxy)ethanol
1-(3-Chlorophenyl)-2-(2,4-diaminoquinazolin-5-yloxy)ethanol
5-[(S)-1-(3-Chlorophenyl)ethoxy]quinazolin-2,4-diamine
2-(2,4-Diaminoquinazolin-5-yloxy)-1-(4-trifluoromethylphenyl)ethanol
2-(4-Chlorophenyl)-2-(2,4-diaminoquinazolin-5-yloxy)ethanol
5-[2-(4-Chlorophenyl)-2-methoxyethoxy]quinazoline-2,4-diamine
5-(2-Methoxy-1-phenylethoxy)quinazoline-2,4-diamine
2-(2,4-Diaminoquinazolin-5-yloxy)-1-(4-fluorophenyl)ethanol
1,1-Bis-(4-chlorophenyl)-3-(2,4-diaminoquinazolin-5-yloxy)butan-1-ol
5-(2-Benzo[1,3]dioxol-5-yl-2-methoxyethoxy)quinazoline-2,4-diamine
2-Benzo[1,3]dioxol-5-yl-2-(2,4-diaminoquinazolin-5-yloxy)ethanol
5-(4-Iodobenzyloxy)quinazoline-2,4-diamine
5-(2,2,2-Trifluoroethoxy)quinazoline-2,4-diamine
5-(4-Chlorobenzyloxy)quinazoline-2,4-diamine
5-(4-Methylbenzyloxy)quinazoline-2,4-diamine
Any of the compounds of Table 1-6 in US2009/0042900
5-{2-[4-(2-Fluoro-benzenesulfonyl)-piperazin-1-yl]-ethoxy}-quinazoline-2,4,diamine
5-{2-[4-(2,4-Difluoro-benzenesulfonyl)-piperazin-1-yl]-ethoxy}-quinazoline-2,4-diamine
5-{2-[4-(3,4-Difluoro-benzenesulfonyl)-piperazin-1-yl]-ethoxy}-quinazoline-2,4-diamine
5-[4-(2-Chlorobenzyl)cyclohexylmethoxy]quinazoline-2,4-diamine
5-[4-(3-Chlorobenzyl)cyclohexylmethoxy]quinazoline-2,4-diamine
5-[4-(2-Fluorobenzyl)cyclohexylmethoxy]quinazoline-2,4-diamine
5-[4-(4-Chlorobenzyl)cyclohexylmethoxy]quinazoline-2,4-diamine
5-(4-Benzyl-piperazin-1-yo)-quinazoline-2,4-di-amine
5-(4-Naphthalen-1-ylmethyl-piperazin-1-yl)-quinazoline-2,4-diamine
[4-(2,4-Diamino-quinazolin-5-yl)-piperazin-1-yo]-naphthalen-1-yl-methanone
5-[4-(Naphthalene-2-sulfonyl)-piperazin-1-yl]-quinazoline-2,4-diamine
5-[4-(2-Methyl-benzyl)-piperazin-1-yl]-quinazoline-2,4-diamine
5-[4-(2,4-Dichloro-benzyl)-piperazin-1-yl]-quinazoline-2,4-diamine
5-[(1-(2-Fluorobenzyl)piperidin-4-ylmethoxy]quinazoline-2,4-diamine
5-[1-(2-Chloro-6-fluorobenzyl)piperidin-4-yl-methoxy]quinazoline-2,4-diamine
5-[1-(2,6-dichlorobenzyl)piperidin-4-ylmethoxy]quinazoline-2,4-diamine

The dosage of compounds of Table 1 or Table 2 depend on several factors, including: the administration method, the severity of the disease, whether the disease is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

Continuous daily dosing with a compound of Table 1 or Table 2 may not be required. A therapeutic regimen may require cycles, during which time a drug is not administered, or therapy may be provided on an as needed basis.

As described above, a compound of Table 1 or Table 2 may be administered orally in the form of a tablet, capsule, elixir, or syrup, or rectally in the form of a suppository. A compound of Table 1 or Table 2 may also be administered topically in the form of a foam, lotion, drop, cream, ointment, emollient, or gel. Parenteral administration of a compound is suitably performed, for example, in the form of a saline solution or with the compound incorporated into liposomes.

The invention features the use of Gem frequency as an indication for treatment of ALS with one or more compounds of Table 1 or Table 2. Moreover, a subject having ALS, or a propensity to develop ALS (e.g., an individual having, or at risk of having, an allele associated with familial ALS), can show an alteration (e.g., a decrease of 10%, 20%, 30%, 40%, 50%, 60%, 70%, 80%, 90%, or more), in Gem frequency compared to a normal reference sample, and will be better candidates for treatment with one or more compounds of Table 1 or Table 2.

The invention also features the use of Gem frequency to determine the dosing of one or more compounds of Table 1 or Table 2 for the treatment of ALS. For example, if a subject's Gem frequency is 10%, 20%, 30%, 40%, 50%, 60%, 70%, 80%, or 90%, less than a normal reference, these patients will be more sensitive to one or more compounds of Table 1 or Table 2 and a lower dose of these compounds can be administered. Desirably, varying the dosing of a compound or compounds of Table 1 or Table 2 based on Gem frequency will provide the optimal treatment to increase Gem frequency in the contacted cells compared to control cells not contacted with the particular compound of Table 1 or Table 2. For example, the dosing of a Table 1 or Table 2 compound can be 50%, 100%, 500%, 1000%, or greater in a subject with a normal frequency of Gems (e.g., a frequency of Gems greater than 50% (e.g., greater than 60%, 70%, 80%, 90%, 95%, or 99%) that of cells isolated from a control subject) compared to dosing of a Table 1 or Table 2 compound for a subject with a decreased frequency of Gems (e.g., a frequency of Gems less than 50% (e.g., less than 40%, 20%, 20%, 10%, 5%, or 1%) that of cells isolated from a control subject).

The invention also features the treatment of ALS as assessed by measurable parameters of improvement in muscle function. One of the earliest symptoms of ALS can include muscle weakness, fasciculation, cramping, stiffness and/or muscle atrophy. A subject having ALS or a propensity to have ALS (e.g., an individual having, or at risk of having, an allele associated with familial ALS) will exhibit these symptoms. Upon diagnosis and treatment based on Gem frequency, a subject having ALS or a propensity to have ALS can show improvement in muscle function (or a decreased rate of muscle function loss) as indicated by electromyography (EMG) or equivalent recording techniques to detect the electrical activity in muscles. The EMG characteristics of improved muscle function can include: an increase in duration of the action potential, an increase in the area to amplitude ratio of the action potential, or an increase in the number of motor units in the muscle as determined using the motor unit number estimation technique. Other symptoms of ALS can include difficulty speaking, slurred speech, and difficulty swallowing. Improvements to these symptoms in a subject receiving treatment can be determined by speech therapy techniques (e.g., articulation therapy, or oral motor therapy), resulting in clear pronunciation of sounds and syllables, and/or ease of swallowing.

### EXAMPLES

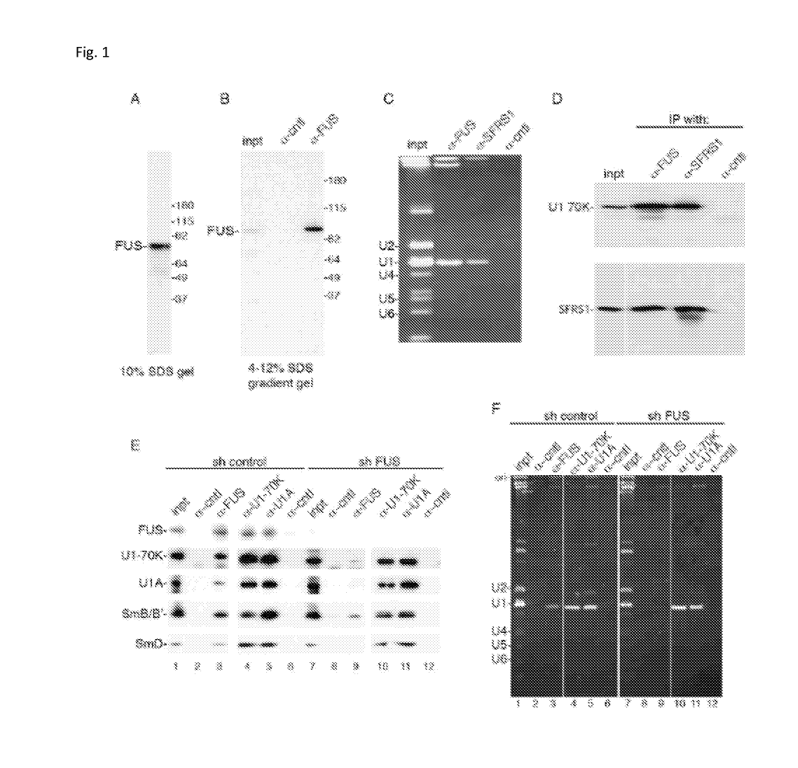

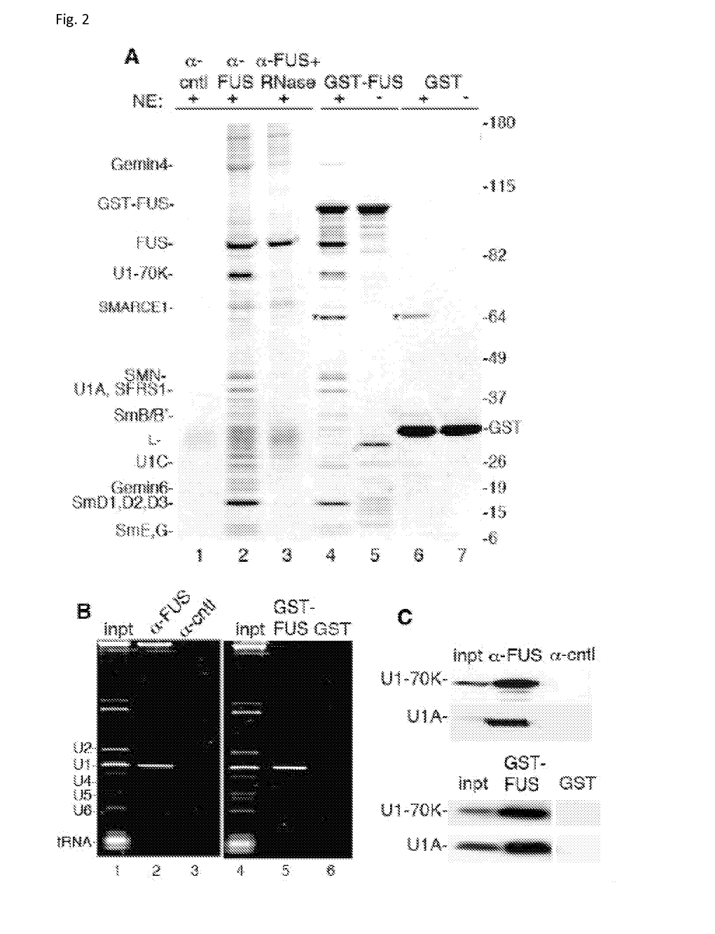

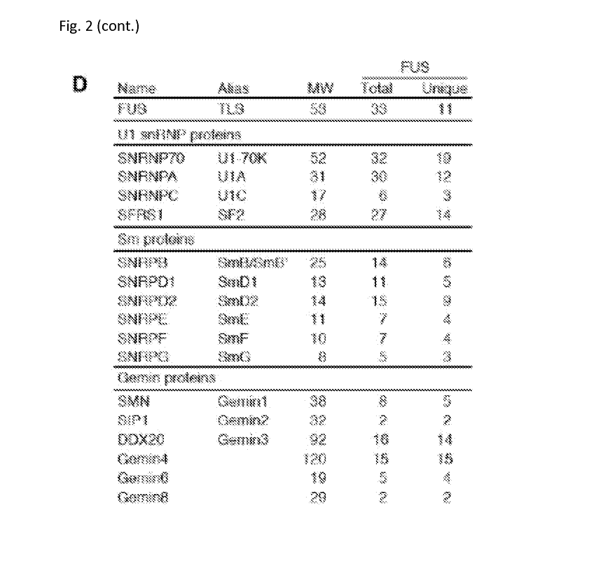

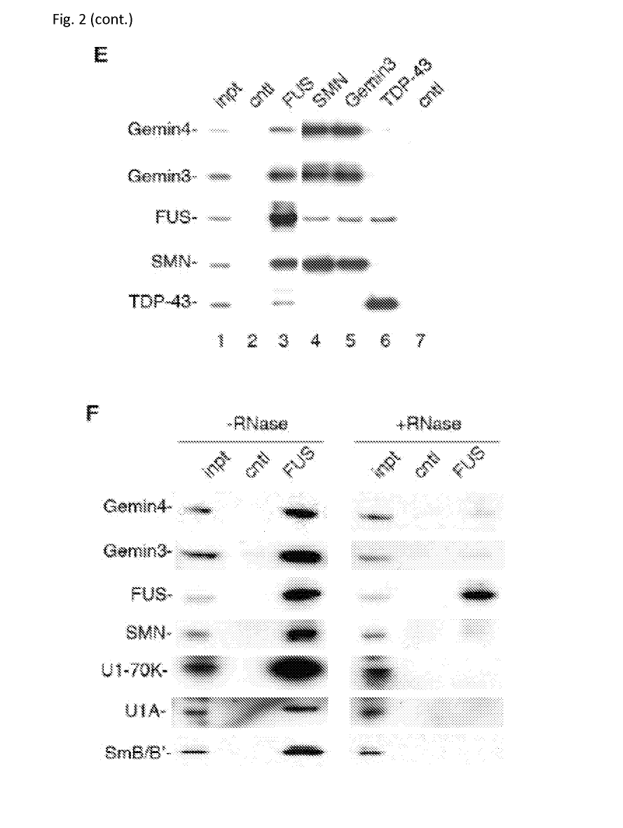

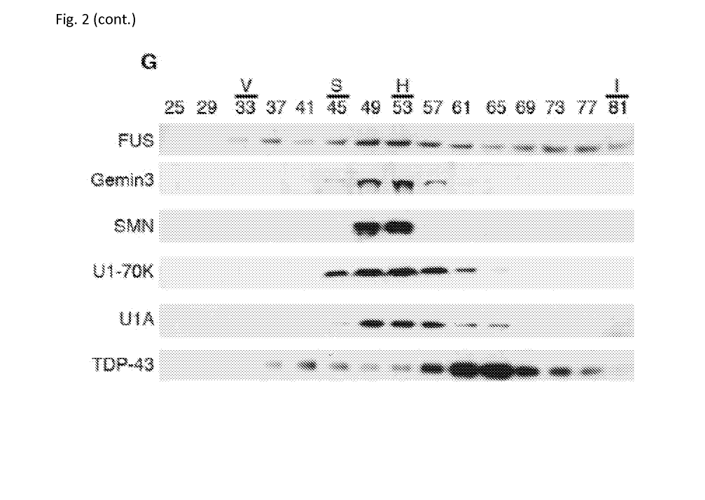

To investigate how mutations in in RNA-related genes cause ALS, we focused on FUS. Antibodies raised against GST-FUS detect one main band by western and immunoprecipitate (IP) FUS from HeLa nuclear extract (FIG. 1). The FUS antibody was used for IPs and GST-FUS was used for pulldowns from nuclear extract, and the proteins were analyzed on a Coomassie gel (FIG. 2A). Mass spectrometry of individual bands revealed that components of U1 snRNP are among the most abundant proteins associated with FUS (FIG. 2A, lanes 2 and 4). In addition, the SR protein SFRS1, which is present in U1 snRNP (FIG. 1) associates with FUS (FIG. 2A, lanes 2 and 4). Finally, U1 snRNA is abundantly detected in both the GST-FUS pulldown and the FUS IP (FIG. 2B), and western analysis confirmed the presence of the U1 snRNP proteins, U1-70K and U1A, in the FUS pulldown and IP (FIG. 2C). As U1 snRNP is known to be an abundant and sticky complex, we further assessed the specificity of its interaction with FUS by carrying out IPs in FUS or scrambled knockdown nuclear extracts (FIG. 1). These data revealed that U1 snRNP components were efficiently IP'd by the FUS antibody only in the scrambled, but not in the FUS, knockdown extract.

Our FUS IP and GST pulldown data also revealed that FUS associates with SMN and the components of the SMN complex (FIG. 2A, D-F; Gemins 2-8). This observation is particularly striking because deficiency in SMN protein levels causes the childhood motor neuron disease SMA. The SMN complex functions in assembly of the spliceosomal snRNPs in the cytoplasm. In the nucleus, the SMN complex is present in Gems, discrete bodies that are not understood but may function in snRNP biogenesis. Previous work showed that the SMN complex interacts directly with U1 snRNA. Consistent with this observation, and with the presence of U1 snRNP in the FUS IP, most of the proteins in the FUS IP are RNase-sensitive (FIG. 2A, lane 3). We further analyzed the proteins associated with FUS by carrying out mass spectrometry of total proteins in FUS and negative control IPs (Table 3). U1 snRNP and Sm snRNP core proteins are the most abundant in the data set (FIG. 2D, Table 3). All of the nuclear components of the SMN complex except for the smallest (Gemin 7, 14 kD) are present in the total FUS IP (FIG. 2D, Table 3). The association of FUS with Gemins was confirmed by IP/western (FIG. 2E). Low levels of Gemins were detected above background when nuclear extract was treated with RNase prior to IP, whereas U1 snRNP components (U1-70K, U1A, SmB/B′) were not (FIG. 2F). We conclude that FUS associates with U1 snRNP in an RNA-dependent manner and with the SMN complex in a largely RNA-dependent manner. The additional proteins detected in the FUS IP (Table 3) may function in other processes ascribed to FUS (Law et al., Brief Funct Genomic Proteomic 5: 8-14 (2006); Zhang et al., Cell 133: 585-600 (2008)).

TABLE 3

| Name | Alias | MW | Unique | Total |
|---|---|---|---|---|
| FUS | TLS | 53 | 11 | 33 |

UI snRAP

| | | | | |
|---|---|---|---|---|
| SNRNP70 | U1-70K | 52 | 19 | 32 |
| SNRPA | U1A | 31 | 12 | 30 |
| SNRPC | U1C | 17 | 3 | 6 |
| SFRS1 | SF2 | 28 | 14 | 27 |

Sm Proteins

| | | | | |
|---|---|---|---|---|
| SNRPB | SmB/B′ | 25 | 6 | 14 |
| SNRPD1 | Sm-D1 | 13 | 5 | 11 |
| SNRPD2 | Sm-D2 | 14 | 9 | 15 |
| SNRPE | Sm-E | 11 | 4 | 7 |
| SNRPF | Sm-F | 10 | 4 | 7 |
| SNRPG | Sm-G | 8 | 3 | 5 |

Gemin proteins

| | | | | |
|---|---|---|---|---|
| SMN | Gemin1 | 38 | 5 | 8 |
| SIP1 | Gemin2 | 32 | 2 | 2 |
| DOX20 | Gemin3 | 92 | 14 | 16 |
| Gemin4 | | 120 | 15 | 15 |
| Gemin6 | | 19 | 4 | 5 |
| Gemin8 | | 29 | 2 | 2 |

SR proteins

| | | | | |
|---|---|---|---|---|
| SFR59 | SRp30c | 26 | 6 | 6 |
| SFR57 | 9G8 | 27 | 5 | 6 |
| SFR53 | SRp70 | 19 | 3 | 3 |
| TRA2B | TRA2β | 34 | 2 | 3 |
| SFR55 | SRp40 | 31 | 2 | 2 |
| TRA2A | TRA2α | 33 | 2 | 2 |

hnRNPs

| | | | | |
|---|---|---|---|---|
| HNRNPR | | 71 | 25 | 40 |
| SYNCRIP | hnRNPQ | 51 | 17 | 24 |
| HNRNPA3 | | 40 | 10 | 16 |
| HNRNPA1 | | 39 | 9 | 14 |
| HNRNPD | AUF1 | 38 | 7 | 12 |
| HNRNPC | | 34 | 6 | 12 |
| HNRNPH3 | | 36 | 8 | 10 |
| HNRNPAB | | 32 | 8 | 9 |
| HNRNPCL1 | | 37 | 6 | 7 |

Other snRNP proteins and RNA binding proteins

| | | | | |
|---|---|---|---|---|
| SF3B1 | SF3b155 | 146 | 16 | 17 |
| SF3B2 | SF3b145 | 100 | 12 | 14 |
| SF2A1 | SF3a120 | 89 | 13 | 15 |
| SF3A3 | SF3a60 | 59 | 7 | 8 |
| SNRPB2 | USB″ | 25 | 4 | 4 |
| SNRPA1 | U2A | 28 | 3 | 4 |
| DHX15 | PRP43 | 91 | 10 | 10 |
| CCNT1 | Cyclin-T | 81 | 13 | 20 |
| SART3 | | 110 | 18 | 19 |
| CDK9 | CTK1 | 43 | 10 | 12 |
| LARP7 | | 67 | 10 | 10 |
| HEXIM1 | | 41 | 6 | 9 |
| MEPCE | BCDIN3 | 74 | 7 | 8 |
| PRPFB | Prp8 | 274 | 16 | 16 |
| SNRNP200 | BRR2, U5-200KD | 245 | 12 | 13 |
| DOX23 | PRP28, U5-100KD | 96 | 7 | 8 |
| TUT1 | RBM21, PAPD2 | 94 | 8 | 8 |
| LSM4 | | 15 | 2 | 2 |
| CDC5L | CDC5 | 92 | 18 | 19 |
| PRPF19 | Prp19 | 55 | 10 | 11 |
| XAB2 | SYF1 | 100 | 8 | 10 |
| CRNKL1 | hCRN, SYF3 | 100 | 8 | 8 |
| AQR | IBP160 | 171 | 8 | 8 |
| BCAS2 | SPF27, DAM1 | 26 | 2 | 3 |
| ACIN1 | ACINUS | 152 | 18 | 21 |
| DHX9 | RHA | 141 | 16 | 16 |
| ZCCHC8 | | 79 | 9 | 11 |
| RBM39 | | 59 | 8 | 8 |
| ZFR | | 117 | 18 | 20 |
| SRRT | ARS2 | 101 | 15 | 20 |
| ELAVL1 | HuR | 36 | 12 | 20 |
| PNN | Pinin | 82 | 9 | 9 |
| RBM7 | | 31 | 7 | 9 |
| G3BP2 | | 54 | 6 | 9 |
| ADAR | | 136 | 7 | 8 |
| SRP14 | ALURBP | 15 | 3 | 3 |
| NPM1 | B23 | 33 | 2 | 3 |
| PABCP1 | PABP1 | 71 | 21 | 29 |
| LARP1 | | 124 | 15 | 19 |
| STRBP | ILF3L | 74 | 10 | 14 |
| CAPRIN1 | GPIAP1 | 78 | 10 | 11 |
| CPSF7 | CFIm59 | 52 | 9 | 9 |
| THOC2 | THO2 | 183 | 9 | 9 |
| DOX39 | URH49 | 49 | 6 | 7 |
| EXOSC4 | RRP41 | 26 | 5 | 5 |
| NCBP2 | CBP20 | 18 | 4 | 5 |
| NUDT21 | CPSF5 | 26 | 3 | 4 |
| SARNP | CIP29 | 24 | 3 | 3 |
| EXOSC7 | RRP42 | 32 | 3 | 3 |
| ERH | DROER | 12 | 2 | 3 |
| WIBG | PYM | 23 | 2 | 2 |
| THOC2 | fSAP24 | 24 | 2 | 2 |
| C1orf77 | SRAG | 26 | 2 | 2 |

Transcription-related proteins

| | | | | |
|---|---|---|---|---|
| RUVBL1 | TIP49B | 51 | 17 | 20 |
| ZNF295 | ZBTB21 | 119 | 15 | 17 |
| RUVBL1 | TIP49A | 50 | 13 | 17 |
| RPAP3 | | 76 | 12 | 15 |
| DDXS | p68 | 69 | 11 | 13 |
| PIH1D1 | NOP17 | 32 | 7 | 9 |
| C19orf2 | RMP | 60 | 7 | 8 |
| DOX17 | p72 | 72 | 7 | 7 |
| PFDN5 | | 15 | 5 | 6 |
| PFDN2 | | 17 | 4 | 5 |
| WDR92 | | 40 | 4 | 5 |
| UXT | ART-27 | 18 | 4 | 4 |
| PDRG1 | | 16 | 3 | 4 |
| POLR2E | RPB5 | 25 | 3 | 4 |
| POLR2H | RPB17, RPB8 | 17 | 7 | 2 |
| SMARCC1 | BAF155 | 123 | 24 | 38 |
| SMARCA4 | BRG1 | 185 | 29 | 34 |
| MLL2 | | 593 | 25 | 26 |
| CCDC6 | | 53 | 19 | 24 |
| ILF2 | NF45 | 43 | 15 | 23 |
| PBRM1 | BAF180 | 193 | 18 | 20 |
| SMARCE1 | BAF57 | 47 | 13 | 16 |
| WDHD1 | CTF4 | 126 | 14 | 15 |
| CHD4 | Mi-2b | 218 | 12 | 14 |
| YAP1 | YAP65 | 54 | 11 | 14 |
| HCFC1 | HCF-1 | 209 | 10 | 10 |
| NCOA6 | | 219 | 9 | 9 |
| SAFB2 | | 107 | 8 | 8 |
| ZMYND8 | RACK7 | 132 | 8 | 8 |
| ZBTB7A | | 61 | 6 | 8 |
| SAFB | | 103 | 6 | 8 |
| ARIO2 | BAF200 | 197 | 6 | 8 |
| ASH2L | | 69 | 6 | 8 |
| SAP1B | | 18 | 4 | 5 |
| BCL7C | | 23 | 3 | 3 |
| DPY30 | Saf19 | 11 | 7 | 7 |
| H2AFV | H2AV | 14 | 2 | 2 |
| H1FX | H1X | 22 | 2 | 2 |
| SET | TAF-Iβ | 33 | 2 | 2 |

Miscellaneous

| | | | | |
|---|---|---|---|---|
| UBR5 | EDD | 309 | 62 | 84 |
| BAT2L | PRRC2B | 243 | 30 | 34 |
| BAT2D1 | PRRC2C, XTP2 | 317 | 22 | 25 |
| AHNAK2 | | 617 | 21 | 22 |
| SBNO1 | MOP3 | 154 | 20 | 22 |
| MPRIP | | 117 | 18 | 21 |
| GOPC | | 51 | 14 | 21 |
| MKI67 | Nopp34 | 34 | 18 | 19 |
| AKAP8 | AKAP95 | 76 | 12 | 19 |
| ABCF3 | | 80 | 14 | 17 |
| BAT2 | PRRC2A | 229 | 14 | 17 |
| PPFIBP1 | SGT2 | 34 | 15 | 15 |
| SEC16A | | 234 | 14 | 14 |
| DNMT1 | | 183 | 12 | 14 |
| CSNK2A2 | CKIIα | 41 | 11 | 14 |
| ZC3H18 | NHN1 | 106 | 10 | 13 |
| CSNK2A1 | | 46 | 10 | 12 |
| SCYL2 | | 104 | 11 | 11 |
| BOD1L | FAM44A | 330 | 10 | 10 |
| CCBL2 | KAT3 | 51 | 8 | 10 |
| GORAB | | 45 | 7 | 10 |
| PALMD | | 63 | 8 | 8 |
| ZNF609 | | 151 | 8 | 8 |
| GPATCH8 | | 164 | 7 | 8 |
| LY6G5B; CSNK2 [symbol] | CKIIβ | 25 | 4 | 6 |
| YWHAZ | 14-3-3 zeta/d [symbol] | 28 | 3 | 3 |
| PDCD6 | AIP1 | 96 | 3 | 3 |
| SKP1 | | 19 | 2 | 2 |
| BOD1 | FAM44B | 19 | 2 | 2 |
| C21orf70 | | 25 | 2 | 2 |
| YWHAG | 14-3-3 gamma | 28 | 2 | 2 |
| KCTD15 | | 32 | 2 | 2 |
| CAPZB | | 32 | 2 | 2 |
| CAPZA1 | | 33 | 2 | 2 |

[symbol]

indicates data missing or illegible when filed

We note that TDP-43 was not significantly detected above background in the total FUS IP/mass spectrometry relative to the negative control IP (Table 3). In light of previous reports that TDP-43 and FUS interact, we used a TDP-43 antibody for IP/westerns, which showed that TDP-43 and FUS co-IP in nuclear extract (FIG. 2E). However, in marked contrast to FUS, TDP-43 does not co-IP with components of the SMN complex or U1 snRNP (FIG. 2E). Consistently, gel filtration of nuclear extract followed by westerns revealed that while both TDP-43 and FUS are present in high-molecular weight complexes, TDP-43 has a profile that is distinct from that of U1 snRNP and the SMN complex but does overlap with FUS (FIG. 2G). These data indicate that although FUS and TDP-43 interact, the two proteins also have distinct molecular associations. Indeed, TDP-43 associates with a large number of hnRNP proteins and components of the miRNA processing machinery.

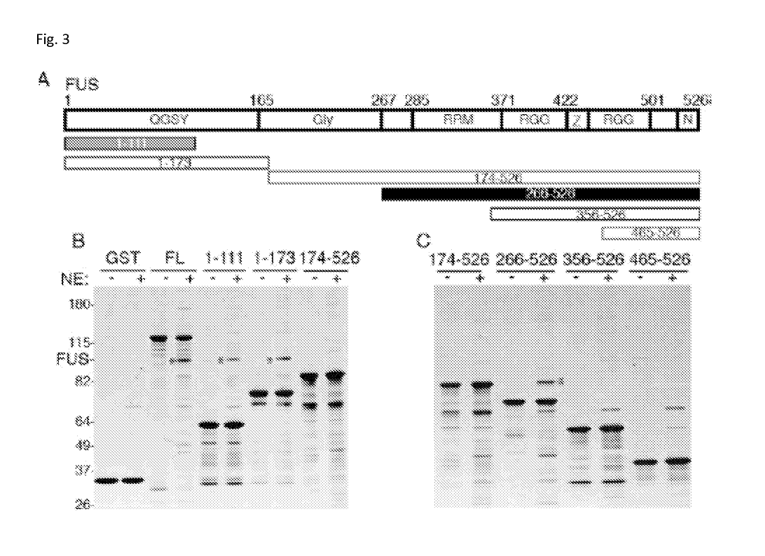

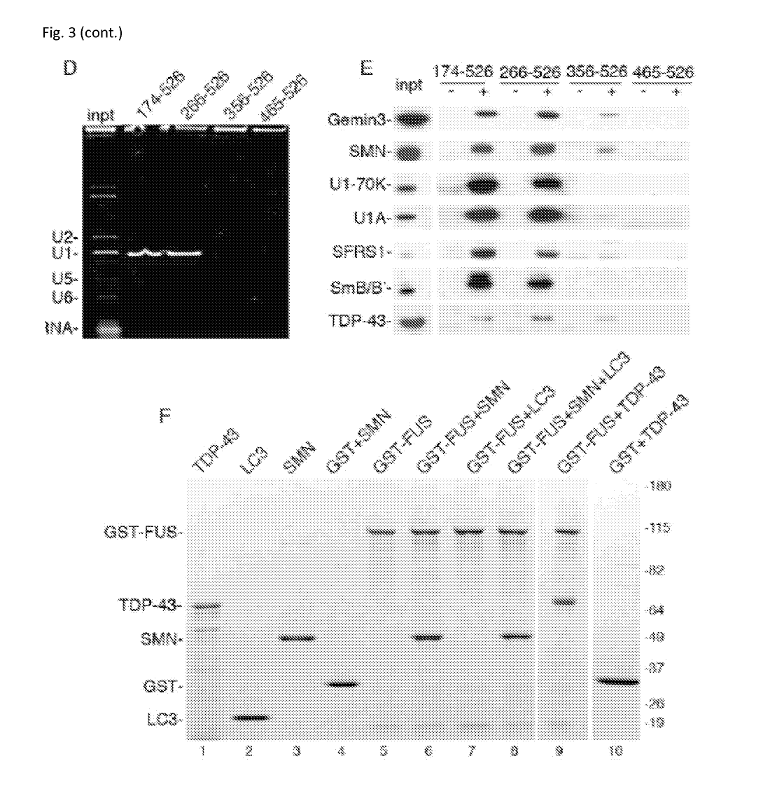

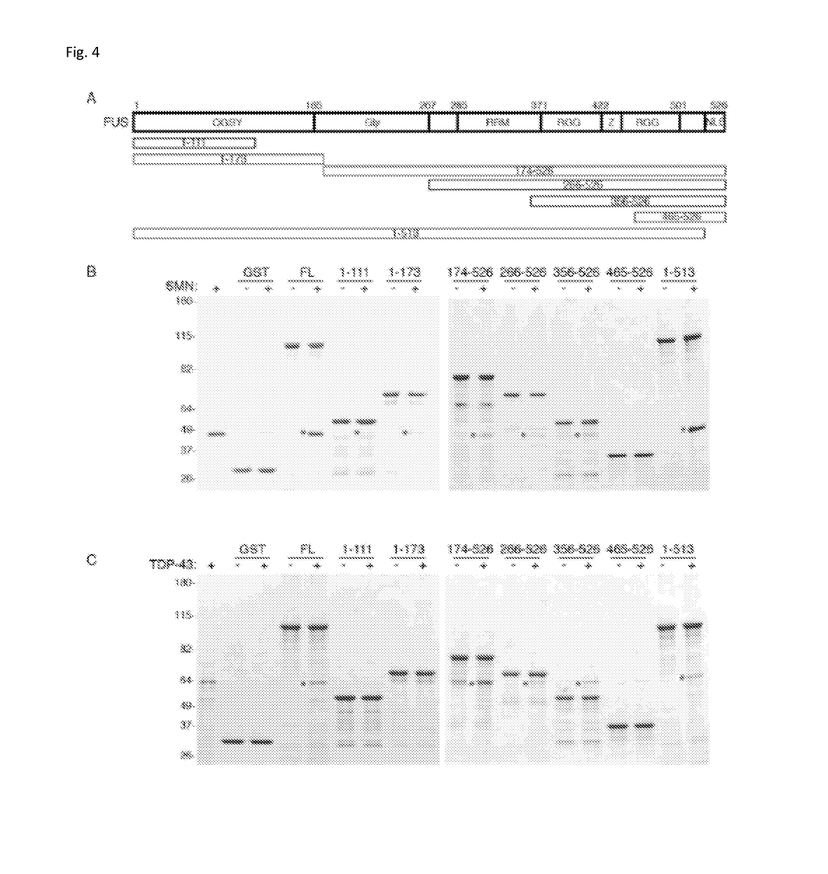

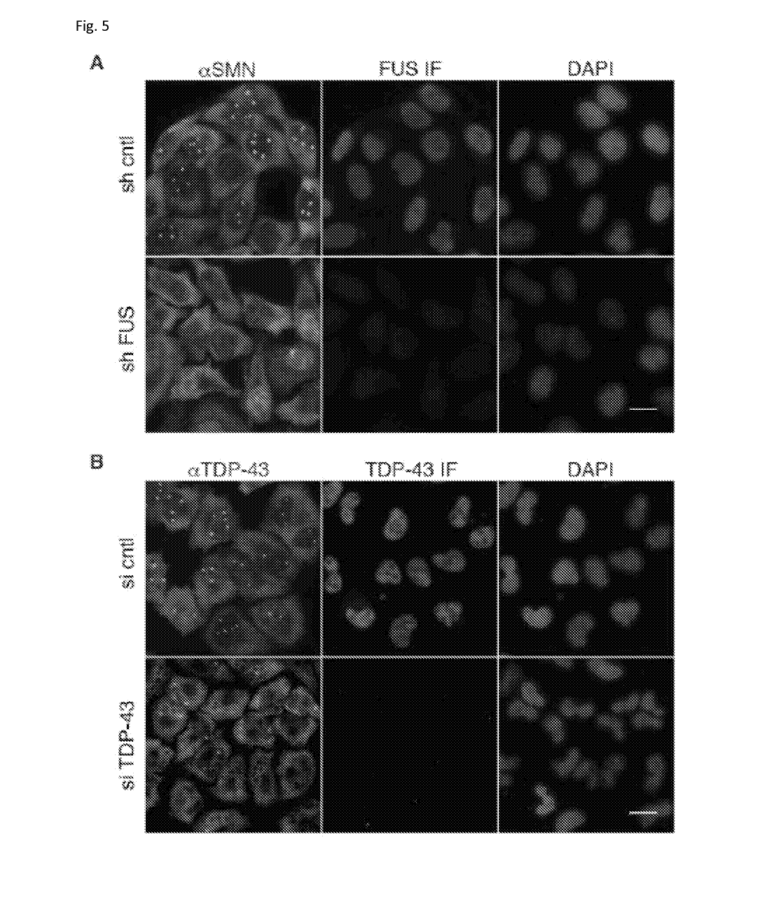

To identify the regions on FUS that interact with the SMN complex and U1 snRNP, we carried out pulldowns from nuclear extract using truncated GST-FUS proteins (FIG. 3A). This analysis revealed that FUS associates with itself, as endogenous FUS present in the nuclear extract binds to full length GST-FUS (FIG. 3B, FL). An amino-terminal region of FUS (1-111) is necessary and sufficient for the FUS-FUS interaction (FIG. 3B, * indicates FUS). The FUS RNA-recognition motif (RRM) is required for the interaction with U1 snRNP, as FUS truncations lacking the RRM (FIG. 3A, 356-526 and 465-526) do not associate with U1 snRNA (FIG. 3D) or with U1 snRNP (FIG. 3E). The interaction between FUS and the SMN complex is also largely lost with FUS truncations lacking the RRM (FIG. 3E). Previous work showed that the SMN complex binds directly to U1 snRNA, which may mediate the RRM-dependent binding of the SMN complex to FUS. In contrast to U1 snRNP, low levels of the SMN complex (SMN, Gemin3) bind to the RRM truncation 356-526 (FIG. 3E), consistent with the finding that the FUS-SMN complex interaction is partially RNA-independent (FIG. 3F). These observations suggest that FUS and SMN might directly interact. To test this, we carried out pulldowns using GST-FUS and purified SMN protein in the presence of RNase. Significantly, full length GST-FUS forms a ˜1:1 complex with SMN but does not associate with negative control proteins (GST and LC3) (FIG. 3F, lanes 2-8). GST-FUS also associates directly with TDP-43 (FIG. 3F, lanes 1, 9, 10). The specificity of the interaction between FUS and SMN and between FUS and TDP-43 was further confirmed using our FUS truncations (FIG. 4). Maximal binding of SMN to FUS requires all but the C-terminus of FUS containing the NLS whereas maximal binding of TDP-43 to FUS requires the RGG domains (FIG. 4). We conclude that both SMN and TDP-43 bind directly to FUS, and distinct regions of FUS are involved.

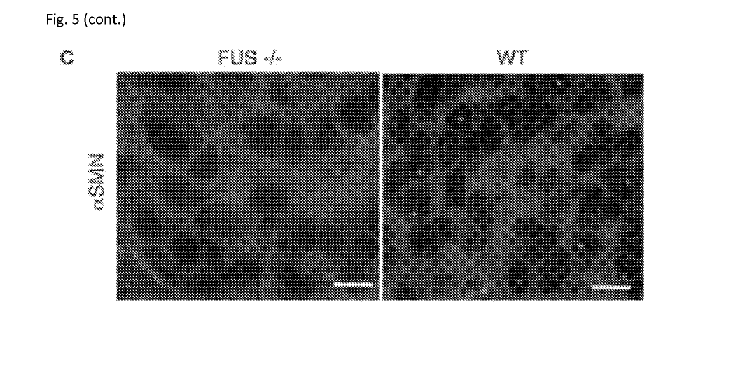

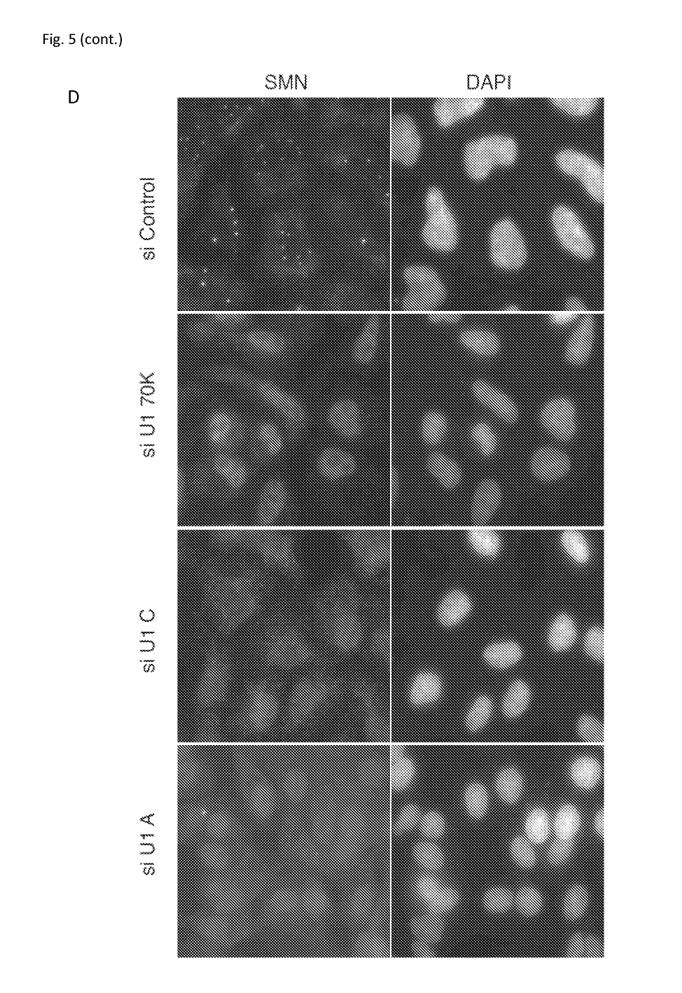

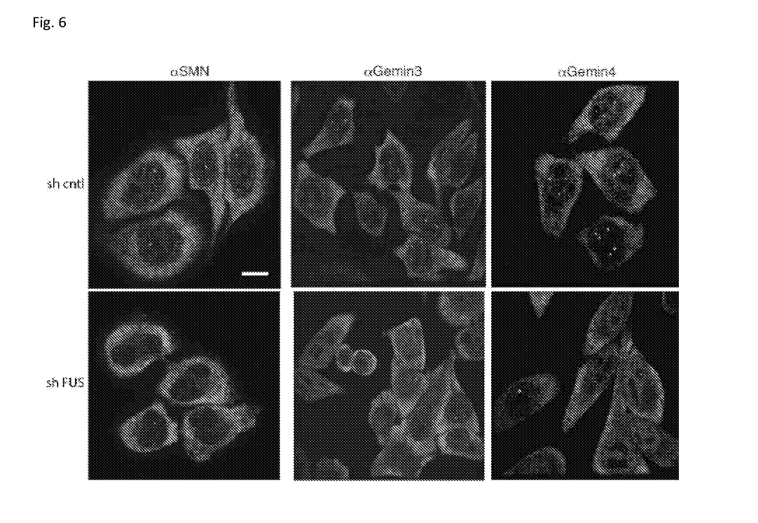

We next asked whether the physical association between FUS and SMN has functional consequences. FUS was targeted with shRNA in HeLa cells using scrambled shRNA as a control, and the SMN and FUS were examined by immunofluorescence (IF). IF of FUS showed that this protein is efficiently knocked down and, as expected, is localized in the nucleus in control cells (FIG. 5A). Strikingly, the SMN IF revealed that the number of nuclear Gems was dramatically reduced in the FUS knockdown cells (FIG. 5A, left panels). The same results were obtained using antibodies to Gemins 3 and 4 (FIG. 6). We conclude that FUS is required for formation and/or stability of Gems. A recent study using a TDP-43 knockout mouse showed that TDP-43 is required for normal Gem levels in spinal cord motor neurons. Consistently, knockdown of TDP-43 showed that it is required for Gems in HeLa cells (FIG. 5B). Moreover, Gems are deficient in FUS−/− spinal cord motor neurons (FIG. 5C). We also found that knockdown of the U1 snRNP proteins, U1-70K, U1A, and U1C, using siRNA, resulted in a lack of Gems in HeLa cells compared to control knockdown cells (FIG. 5D). We conclude that FUS and TDP-43 are required for the formation and/or stability of Gems in HeLa cells and in mouse spinal cord motor neurons.

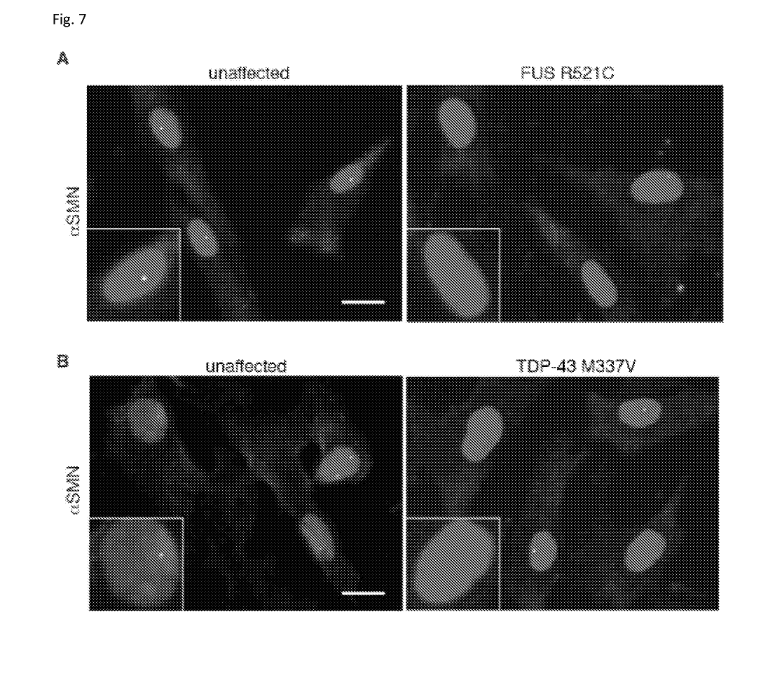

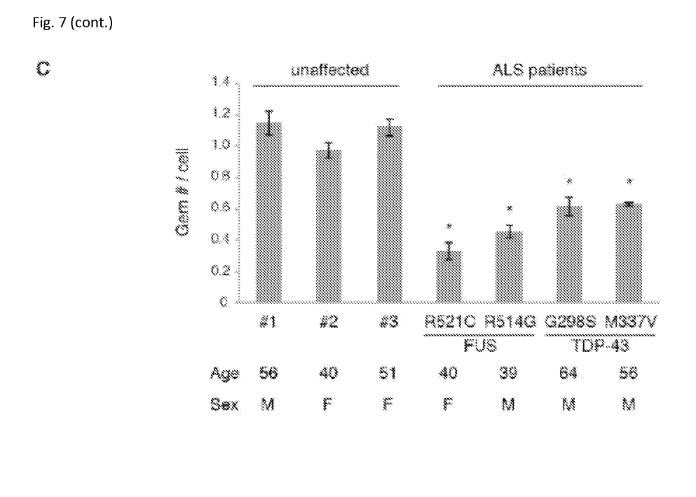

Previous work showed that Gem levels are reduced in SMA patient fibroblasts, with the severity of disease correlating with the reduction in Gems. We therefore examined Gems in ALS patient fibroblasts bearing a FUS R521C or TDP-43 M337V mutation compared to age- and sex-matched fibroblasts from unaffected individuals. Strikingly, Gem deficiency was observed in both FUS and TDP-43 patient cells compared to controls (FIGS. 7A and B). To confirm and extend these results, we used an automated system to collect images from three biological replicates of each fibroblast and then counted Gems in a total of more than 800 cells for each. We also used this system to examine fibroblasts from an additional FUS (R514G), TDP43 (G298S), and unaffected individual. These data revealed that the average Gem number was about 2-3 fold lower in the FUS fibroblasts and about 1.8 fold lower in the TDP-43 fibroblasts relative to controls (FIG. 7C). We conclude that a single amino acid change in FUS or TDP-43 that causes ALS results in a Gem deficiency in patient fibroblasts.

Experimental Procedures

Plasmids and Proteins

GST-FUS and truncations were constructed by inserting a PCR fragment containing full length or portions of FUS into the BamHI and XhoI sites of pGEX-6P-1. Purified His-SMN and His-TDP-43 were from Enzo Life Sciences and Proteintech, respectively.

Antibodies

Rabbit polyclonal antibodies were raised against GST-FUS (Covance). Antibodies to SMN (2B1), Sm (Y12), Gemin3 (12H12), and Gemin4 were from Abcam, U1-70K (9C4.1) and SFRS1 (AK96) from Millipore, TDP43 from Proteintech, and U1A (BJ-7) and HA from Santa Cruz. SAP130 and HA were used as negative controls for polyclonal and monoclonal antibodies, respectively.

Mass Spectrometry

FUS and control IPs were TCA-precipitated and analyzed by LC-MS/MS. Gel samples were trypsin digested and peptides analyzed by LC-MS/MS. Keratin and abundant proteins likely to be contaminants, such as, desmoplakin, actin, tubulin, myosin, and translation proteins were not included in Table 3. Proteins found in the negative control IP were not included in Table 3 if the total peptides were less than 3 fold lower than in the FUS IP. Proteins greater than 300 amino acids for which only 7 peptides or less were identified by mass spectrometry were not included in Table 3. Proteins with less than 2 unique peptides were also omitted.

RNAi

Lentivirus-mediated shRNA was used against FUS

(CCGGCGTGGTGGCTTCAATAAATTTCTCGAGAAATTTATTGAAGCCACCACGTTTTT, Open Biosystems) with a scrambled negative control (CCTAAGGTTAAGTCGCCCTCGCTCGAGCGAGGGCGACTTAACCTTAGG, Addgene). Lentiviruses were prepared according to manufacturer's instructions (ViraPower™ Lentiviral Expression System, Invitrogen). TDP-43 siRNA (ON-TARGET SMARTpool) and scrambled control were from Thermo Scientific.

Immunofluorescence (IF)

IF was carried out using antibodies FUS (1:1000), TDP-43 (1:1000), SMN (1:400), Gemin3 (1:400), and Gemin4 (1:400).

IF and Gem Imaging

HeLa cells were fixed with 4% paraformaldehyde in PBS for 15 min, and fibroblasts were fixed with methanol and acetone (1:1) for 15 min Cells were permeabilized with 0.1% TritonX-100 in PBS for 15 min For IF, cells were incubated in primary (10) antibody overnight at 4° C. After 3 washes in PBS, 20 antibody was added for 1 hr at RT, followed by 3 washes in PBS. 10 antibodies FUS (1:1000), TDP-43 (1:1000), SMN (1:400), Gemin3 (1:400), and Gemin4 (1:400) were diluted in 10% calf serum in PBS. 20 antibodies were mouse Alexa-488 and rabbit Alexa-647 diluted 1:1000 in 10% calf serum in PBS. For HeLa cells, images were captured with a Nikon TE2000U inverted microscope with a PerkinElmer ultraview spinning disk confocal and a 20X objective using Metamorph software (Molecular Devices, Sunnyvale, Calif.). For mouse motor neurons, spinal cords were dissected, post-fixed for 2 hr, vibratome sectioned (Leica) and stored at 4° C. Sections (50 μm) were permeabilized and blocked overnight at 4° C. in 1% Triton X-100, 4% BSA and incubated for 24 hr with SMN antibody (1:250). Sections were washed for 2 hr in PBS, and incubated for 24 hr with Alexa-594 20 antibody (1:1000). After washing for 2 hr in PBS, spinal cord sections were mounted in Vectashield (Vector Labs) and visualized using a laser scanning confocal microscope (Olympus FV-1000). Images were obtained with a 60X P1anAPO (1.4 NA) oil immersion objective. Image stacks (z=0.35 μm) were projected in 2D using Fluoview (Olympus) and processed in Adobe Photoshop (CS4). For automated imaging, fibroblast images were captured with a Nikon Ti motorized inverted microscope using a 20X, Plan Apo 0.75 NA objective. Images were acquired with a Hammamatsu ORCA-R2 cooled CCD camera (widefield) with Metamorph 7 software (Molecular Devices, Sunnyvale, Calif.). For each field, four z-series optical sections were collected with a step size of 0.5 μm. A Prior Proscan motorized stage and Metamorph software was used to automate collection of 40-60 non-overlapping fields of view from each slide. Final images were displayed as maximum z-projections.

### Other Embodiments

The description of the specific embodiments of the invention is presented for the purposes of illustration. It is not intended to be exhaustive or to limit the scope of the invention to the specific forms described herein. Although the invention has been described with reference to several embodiments, it will be understood by one of ordinary skill in the art that various modifications can be made without departing from the spirit and the scope of the invention, as set forth in the claims. All patents, patent applications, and publications referenced herein are hereby incorporated by reference.

What is claimed is:

1. A method of diagnosing a subject as having, or at risk of developing, ALS, said method comprising obtaining a sample of non-neuronal cells, or progeny thereof, isolated from said subject and determining the frequency of Gems in the isolated cells, or progeny thereof, wherein a decreased frequency of Gems is indicative of the subject having, or being at risk of developing, ALS.

2. The method of claim 1, wherein a frequency of Gems less than 50% that of cells isolated from a control subject, is indicative of the subject having, or being at risk of developing, ALS.

3. The method of claim 1, wherein a frequency of Gems less than an average of 1 per cell is indicative of the subject having, or being at risk of developing, ALS.

4. (canceled)

5. The method of claim 1, wherein the non-neuronal cell sample comprises cells selected from the group consisting of epithelial cells, fibroblasts, fibrocytes, myocytes, tendon cells, myocardiocytes, adipocytes, interstitial cells, lymphocytes, gastric chief cells, parietal cells, goblet cells, hepatocytes, urothelial cells, osteocytes, and paneth cells.

6. The method of claim 1, wherein the non-neuronal cell sample is present in urine, blood, serum, plasma, saliva, amniotic fluid, or cerebrospinal fluid.

7. The method of claim 1, wherein said frequency of Gems is determined using confocal microscopy.

8. The method of claim 1, wherein said frequency of Gems is determined using a fluorescent microscope.

9. The method of claim 1, wherein said frequency of Gems is determined by contacting said cell sample with an antibody specific for a Gem protein or a probe specific for a Gem snRNA.

10.-12. (canceled)

13. A method of identifying a candidate compound that treats ALS, said method comprising the steps of (a) contacting cells with a candidate compound, (b) comparing the frequency of Gems in the contacted cells with the frequency of Gems in cells not contacted with said candidate compound, and (c) identifying a compound which increases the frequency of Gems in said contacted cells compared to said cells not contacted with said candidate compound; wherein an increase in the frequency of Gems is indicative of a compound that treats ALS.

14. The method of claim 13, wherein said cells have decreased FUS or SMN expression prior to step (a).

15. The method of claim 13, wherein said cells have a reduced frequency of Gems prior to step (a).

16. The method of claim 13, wherein said the frequency of Gems is determined using a confocal microscope.

17. The method of claim 13, wherein said frequency of Gems is determined using a fluorescent microscope.

18. The method of claim 13, wherein said frequency of Gems is determined by contacting said cell sample with an antibody specific for a Gem protein or a probe specific for a Gem snRNA.

19. The method of claim 18, wherein said antibody specific for a Gem protein is selected from the group consisting of an anti-SMN, anti-gemin 2, anti-gemin 3, and anti-gemin 4 antibody.

20. The method of claim 13, wherein said frequency of Gems is an average number of Gems per cell.

21. The method of claim 13, wherein said frequency of Gems is an average number of Gems per nucleus.

22. A method of treating ALS in a subject, said method comprising administering to said subject a compound of Table 1, wherein said compound of Table 1 increases the frequency of Gems relative to control in a suitable assay (e.g., using human non-neuronal cells such as fibroblasts).

23. A method of treating ALS in a subject having a decreased frequency of GemS, said method comprising:
determining the frequency of Gems in a sample of non-neuronal cells, or progeny thereof, isolated from said subject, and
administering to said subject a compound of Table 1 or Table 2 if said frequency of GemS is decreased.

24.-29. (canceled)

30. The method of claim 22, wherein said treating of ALS in a subject results in improved muscle function, decreased rate of muscle function loss, decreased difficulty speaking, decreased slurred speech, or decreased difficulty swallowing.

31. A method of diagnosing a subject as having or being at risk for developing ALS, said method comprising obtaining sample from said subject, determining the activity of one or more genes of the SMN complex or U1 snRNP complex in said sample, wherein a decreased activity of one or more genes of the SMN complex or U1 snRNP complex is indicative of a subject having or being at risk for developing ALS.

32. The method of claim 31, wherein said one or more genes of the SMN complex are selected from the group consisting of SMN, Gemin2, Gemini, Gemin4, Gemin5, Gemin6, Gemin7, Gemin8, and UNRIP.

33. (canceled)

34. The method of claim 31, wherein said one or more genes of the U1 snRNP complex are selected from the group consisting of U1-70K, U1A, U1C, and U1 snRNA.

35. The method of claim 31, wherein said determining the activity of said one or more genes comprises determining whether said one or more genes is mutated.

36. The method of claim 31, wherein said determining the activity of said one or more genes comprises determining whether the mRNA or protein corresponding to said one or more genes is decreased.

37.-38. (canceled)
